SEQUENCE LISTING (9 sequences)

SEQ ID NO: 1 - skipped

SEQ ID NO: 2 - protein, 15 aa, synthetic construct
AARRSYVSSGEMMVG

SEQ ID NO: 3 - protein, 15 aa, synthetic construct
RHVREAASYQEALAR

SEQ ID NO: 4 - protein, 15 aa, synthetic construct
RLEEEGQSLKDEMAR

SEQ ID NO: 5 - protein, 15 aa, synthetic construct
IRETSLDTKSVSEGH

SEQ ID NO: 6 - protein, 15 aa, synthetic construct
ETSLDTKSVSEGHLK

SEQ ID NO: 7 - protein, 432 aa, synthetic construct
MERRRITSAARRSYVSSGEMMVGGLAPGRRLGPGTRLSLARMPPPLPTRVDFSLAGALNAGFKETRASERAEMMELNDRFASYIEKVRFLEGGNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 8 - protein, 432 aa, synthetic construct
MERRRITSAARRSYVSSGEMMVGGLAPGRRLGPGTRLSLARMPPPLPTRVDFDLAGALNAGFKETRASERAEMMELNDRGASYIEKVRFLEQQNKALAAELNQLRAKEPTKLADVYQAELRELRLRLDQLRANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 9 - protein, 432 aa, synthetic construct
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLEFKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDKKNVKLALDIEIATYRKLLEGEENRITIPVQTFSNLQIRETSLDTKSVSEGHLKRNIVVKTVEMRDGEVIKESKQEHKDVM

(19) United States
(12) Patent Application Publication
(10) Pub. No.: US 2025/0041270 A1
(43) Pub. Date: Feb. 6, 2025

(54) SMALL MOLECULE DRUGS THAT REDUCE PROTEIN AGGREGATION

(71) Applicant: BIOVENTURES, LLC, Little Rock, AR (US)

(72) Inventors: Meenakshisundaram BALASUBRAMANIAM, Little Rock, AR (US); Srinivas AYYADEVARA, Little Rock, AR (US); Robert J. SHMOOKLER REIS, Little Rock, AR (US); Sue GRIFFIN, Little Rock, AR (US)

(21) Appl. No.: 18/719,745

(22) PCT Filed: Dec. 13, 2022
(86) PCT No.: PCT/US2022/081454

§ 371 Date: Jun. 13, 2024

Related U.S. Application Data
(60) Provisional application No. 63/288,998, filed on Dec. 13, 2021.

(51) Int. Cl. A61K 31/402; A61P 25/28

(52) U.S. Cl. CPC A61K 31/402; A61P 25/28

(57) ABSTRACT

Disclosed herein are small molecule drugs that reduce protein aggregation and their methods of use. One aspect of the invention provides for a method reducing aggregate protein abundance in a protein aggregate, the method comprising administering an effective amount of a compound that stably binds a Glial Fibrillary Acidic Protein (GFAP) to a subject, wherein the aggregate protein comprises BSN, SYN1, MAP2, PLEC, RAB10, MAP1A, DCTN, TUBA4A, SPART, PRKDC, or any combination thereof.

### CROSS REFERENCE TO RELATED APPLICATIONS

This application claims the benefit of priority of U.S. Provisional Patent Application Ser. No. 63/288,998, filed Dec. 13, 2021, the contents of which is incorporated by reference in its entirety.

### STATEMENT REGARDING FEDERALLY SPONSORED RESEARCH

This invention was made with government support under P01AG01241117A1 awarded by the National Institutes of Health and Merit 2 101 BX001655 awarded by the Department of Veteran Affairs. The government has certain rights in the invention.

### REFERENCE TO AN ELECTRONIC SEQUENCE LISTING

The contents of the electronic sequence listing (169852.00108.xml; Size: 9,341 bytes; and Date of Creation: Dec. 12, 2022) is herein incorporated by reference in its entirety.

### BACKGROUND

Glial Fibrillary Acidic Protein (GFAP) is a type III intermediate filament structural protein predominantly found in astrocytes, that has been implicated in several age-related neuropathologies. GFAP was shown to be associated with progressive neurological disorders such as Alzheimer's, Parkinson's, and Alexander's diseases (Helman et al., 2020; Ishiki et al., 2016; Kamphuis et al., 2012; Lee et al., 2017; Middeldorp and Hol, 2011; van Bodegraven et al., 2021). Mutation of GFAP, leading to aggregation of Rosenthal fibers, is both causal and diagnostic for Alexander's disease (Lee et al., 2017). GFAP expression is regulated at both the transcriptional and post-translational levels, impacting critical cytoskeletal functions. GFAP is transcriptionally modulated by multiple growth factors and nuclear hormone receptors (Laping et al., 1994). Observed post-translational modifications (PTMs) of GFAP include site-specific phosphorylations by multiple kinases (Battaglia et al., 2019; Clairembault et al., 2014; Herskowitz et al., 2010; Leal et al., 1997; Sullivan et al., 2012), acetylation in amyotrophic lateral sclerosis (ALS) (Liu et al., 2013), and citrullination at 5 arginine residues in AD (Ishigami et al., 2015). The resultant alterations of the GFAP structural conformation may contribute to traumatic brain injury (Lazarus et al., 2015) or autoimmune disorders (Jin et al., 2013). Several single-nucleotide polymorphisms in GFAP are strongly associated with Alexander disease (Lee et al., 2017).

As a result, there exists a need for new drugs that target GFAP for treatment of neurological disorders, such as Alzheimer's, Parkinson's, and Alexander's diseases, as well as related dementias thereof.

### SUMMARY OF THE INVENTION

Disclosed herein are small molecule drugs that reduce protein aggregation and their methods of use. One aspect of the invention provides for a method reducing aggregate protein abundance in a protein aggregate, the method comprising administering an effective amount of a compound that stably binds a Glial Fibrillary Acidic Protein (GFAP) to a subject, wherein the aggregate protein comprises BSN, SYN1, MAP2, PLEC, RAB10, MAP1A, DCTN, TUBA4A, SPART, PRKDC, or any combination thereof. In some embodiments, the subject suffers from a neurodegenerative disorder, such as Alzheimer's disease.

Another aspect of the invention provides for increasing aggregate protein abundance in a protein aggregate, the method comprising administering an effective amount of a compound that stably binds a Glial Fibrillary Acidic Protein (GFAP) to a subject. In some embodiments, the aggregate protein comprises CBX8, TSPYL5, CDK2, KRT33B, or any combination thereof.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly-identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1C show that GFAP is more abundant and more modified in AD aggregates than in AMC. (FIG. 1A) Spectral counts for GFAP in sarkosyl-insoluble aggregates isolated by immuno-pulldown (IP) of A3 or tau, or in total aggregates without IP, are more abundant in human AD than in age-matched-control (AMC) hippocampus. AD differs from AMC by heteroscedastic t tests: *P<0.05; **P<0.005. (FIG. 1B) Differential post-translational modifications (PTMs: phosphorylation of Ser or Thr residues, or oxidations of Met) observed in GFAP isolated from AMC [ApoE(3,3)](SEQ ID NO: 7), AD [ApoE(3,3)](SEQ ID NO: 8), or AD [ApoE(4,4)](SEQ ID NO: 9) hippocampus, as indicated. Peptide coverage is indicated by highlighting. (FIG. 1C) Western blot of phosphorylated GFAP from AMC(3,3) vs. AD(3,3) or AD(4,4) hippocampal aggregates, detected with antibody to phospho-GFAP (Ser13; ThermoFisher). ***Each AD group differs from AMC by 2-tailed heteroscedastic t test at P<0.0001.

FIGS. 2A-2E illustrate molecular-dynamic analyses of GFAP structure. (FIG. 2A) Initial modelled structure of GFAP. The internal cavity is the predicted drug-binding pocket. (FIG. 2B) Predicted binding pocket volume for GFAP at 50-nsec intervals over a 500-nsec span. (FIG. 2C) Predicted cavity for ligand binding at 200 nsec. (FIG. 2D) Time course of root mean squared deviation (RMSD) for GFAP structure, comparing 500-nsec in silico simulations of AMC (unmodified) GFAP to simulations of GFAP with phosphomimetic substitutions to mimic AD(3,3) and AD(4,4). (FIG. 2E) Distribution across GFAP (432 residues) of root mean squared fluctuation (RMSF), to illustrate positional variability by residue. (FIG. 2D, FIG. 2E) Keys to the right of FIGS. 2D and 2E display the color codes for RMSD and RMSF values, respectively.

FIGS. 3A-3E illustrate effects on aggregation of RNAi knockdowns targeting GFAP or its putative kinases. (FIG. 3A) Observed GFAP phosphorylations in human hippocampal aggregates, and their putative kinases. (FIG. 3B) Fluorescence images of Thioflavin T-stained human SH-SY5Y-APP_{Sw} cells, after liposome-mediated transfection with siRNA constructs targeting GFAP or its candidate kinases. (FIG. 3C) Histogram showing means±SEM for Thioflavin T staining per cell, of aggregates stained as in panel 3b. (FIG. 3D) Thioflavin T-stained human T98G cells, after transfection with siRNA constructs as shown. (FIG. 3E) Histogram of means±SEM for Thioflavin T staining of aggregates as in panel 3d. (FIG. 3C-3E) Numbers over bars are P values for differences between treated groups and controls, by 2-tailed t tests. Numbers over brackets apply to the groups/bars connected.

FIGS. 4A-4B show that ROCK1 protein levels are higher in T98G glioblastoma cells overexpressing an ApoE4 transgene than in T98G cells overexpressing ApoE3. (FIG. 4A) Western blot probed with antibody to ROCK1 protein. (FIG. 4B) Mean ±SEM of western-blot band intensity for independent T98G cell cultures (each N=5). E4 >E3 at P<0.0001 by t test.

FIGS. 5A-5F illustrates that the drug MSR1 is predicted to specifically bind GFAP and thereby block its role in aggregation. (FIG. 5A) Histogram indicating the stability (Gibbs free energy of binding) of GFAP binding predicted by MM-GBSA solvated docking of the top 3 drugs from in silico screening. The ChemBridge drug-structure library was screened for binding to GFAP, in 3 stages of progressively increased stringency, followed by counter-screening to eliminate drugs with affinity for tubulins. (FIG. 5B) SY5Y-APPs, cells were stained with Thioflavin T (green fluorescence) and counterstained with DAPI (not shown). (FIG. 5C) Histogram showing 2-fold reduction in amyloid per cell (thioflavin-T fluorescence divided by the number of DAPI+nuclei per field, as illustrated in FIG. 5B; ***P<0.0005. (FIG. 5D) Total sarkosyl-insoluble aggregate protein, stained with SYPRO-Ruby following isolation from SY5Y-APP_{Sw} cells. (FIG. 5E) The set of proteins totally removed from aggregates by siRNA knockdown of GFAP is nearly identical to the set eliminated by MSR1. The Venn diagram shows proteomic overlap of 251 proteins identified in sarkosyl-insoluble aggregates from untreated SYSY-APP_{Sw} cells (>7 hits), but not detected 48 hr after transfection with GFAP siRNA, or treatment with 1-μM MSR1. Conversely, 4 proteins absent from untreated-cell aggregates were identified in both treated cell groups. (FIG. 5F) Linear regression of log₂(fold change) of aggregate protein abundance after GFAP siRNA treatment (x axis) vs. MSR1 treatment (y axis). Selected proteins are labeled, including those that were shown by aggregate cross-linking (Balasubramaniam et al., 2019) to be immediate neighbors of GFAP (red dots including BSN, SYN1, MAP2, PLEC, RAB10, MAP1A, DCTN, TUBA4A, SPART, PRKDC, EEF2, PARP1, NFH, and H14). Dots within the dashed rectangle were shifted <2-fold by either treatment. R=0.77 for the regression; significance by F test was P<3E-280.

FIG. 6 shows the molecular structures of MSR-1, MSR-2, and MSR-3.

FIGS. 7A-7F illustrate MSR1 treatment attenuates aggregates and its associated phenotypic traits in C. elegans models of AD. (FIG. 7A) RNAi knockdown of BARK or ROCK] in C. elegans strain AM141 (a model of Huntington's disease with muscle expression of Q40::YFP), is compared to the effect of RNAi targeting IFP-1, the closest C. elegans homolog of GFAP. (FIG. 7B) RNAi knockdown of ROCK1, BARK or AKT2, in C. elegans strain CL2355 (an AD model with pan-neuronal expression of human Aβ₁₋₄₂). (FIG. 7C) RNAi knockdown of ROCK1, AKT2 or BARK, in human T98G cells, reduces the total aggregate burden (measured as total spectral counts for sarkosyl-insoluble material). (FIG. 7D) MSR1 protects against aggregation of tau, expressed in muscle of C. elegans strain VH255. (FIG. 7E) Chemotaxis of C. elegans strain CL2355, an AD model with pan-neuronal expression of human Aβ₁₋₄₂. Chemotaxis to n-butanol was determined for adult worms on day 5 post-hatch. (FIG. 7F) Calculated average aggregate fluorescence per worm in C. elegans strain AM141 (q40::yfp), a model of Huntington's disease featuring muscle expression of polyglutamine (Q40) fused in-frame to the gene encoding yellow fluorescent protein (YFP). YFP-positive aggregates were counted in adult worms on day 5 post-hatch. (FIG. 7A-7F) Differences from controls are significant by heteroscedastic, 2-tailed t tests at *P≤0.05; **P≤0.005; ***P≤0.0005; ****P≤0.00005. Experiments were replicated 3-4 times, with consistent results.

### DETAILED DESCRIPTION OF THE INVENTION

Disclosed herein are small-molecule drugs that target GFAP and reduce protein aggregation. The Examples demonstrate that GFAP is markedly overexpressed and differentially phosphorylated in AD hippocampus, especially in AD subjects, of the apolipoprotein E [F4, F4] genotype, ApoE(4,4), relative to age-matched controls (AMCs). Hyperphosphorylated GFAP is particularly enriched in detergent-insoluble aggregates of AD brain, over AMC. Four kinases that could be responsible for the observed site-specific phosphorylations of GFAP in AD brain (ROCK1, BARK/GRK, PKA, and AKT2) are upregulated in AD relative to AMC, and knockdown of these kinases in SH-SY5Y-APP_{Sw} human neuroblastoma cells or T98G human glioblastoma cells significantly reduced amyloid accumulation. Knockdown of the orthologous kinases in C. elegans also diminished protein aggregation and its associated behavioral traits, in several models of Alzheimer's-like aggregation and in a model of polyglutamine aggregation as observed in Huntington's disease. In silico screening identified a drug candidate, termed MSR1, with stable and specific binding to GFAP. Cell aggregates are reduced to a similar extent by MSR1 exposure or GFAP-specific RNAi knockdown, with remarkably high concordance of aggregate proteins depleted. The Examples show that GFAP plays a key role in neuropathic aggregate accrual. It is a functional target, a valuable biomarker, and a novel therapeutic target to prevent or alleviate AD and other neurodegenerative disorders.

Glial Fibrillary Acidic Protein (GFAP) is a type-III intermediate filament structural protein predominantly found in astrocytes, which may play causal roles in several age-related neuropathologies (Hol and Capetanaki, 2017). GFAP has been implicated functionally in animal models of AD (Kamphuis et al., 2012) and Alexander's disease (Helman et al., 2020; Lee et al., 2017). Detection of GFAP in human CSF or serum is a critical biomarker of neuropathology, contributing to diagnoses of Alzheimer's disease (AD), Parkinson's disease (PD), dementia with Lewy bodies (DLB) and frontotemporal lobar degeneration (FTLD) (Bartd et al., 2021; Ishiki et al., 2016; Schulz et al., 2021).

GFAP mutations, favoring aggregation to form Rosenthal fibers, are both causal and diagnostic for chronic gliosis as found in Alexander's disease (Lee et al., 2017), and several single-nucleotide polymorphisms in GFAP are strongly associated with this demyelination disorder (Lee et al., 2017). GFAP expression is transcriptionally modulated by multiple growth factors and nuclear hormone receptors (Laping et al., 1994). Post-translational modifications (PTMs) previously reported for GFAP include site-specific phosphorylations associated with Alexander's disease (Battaglia et al. 2019), PD (Clairembault et al., 2014), and FTLD (Herskowitz et al., 2010); acetylations at 6 lysine residues in amyotrophic lateral sclerosis (ALS) (Liu et al., 2013); and citrullination at 5 arginine residues in AD (Ishigami et al., 2015). Citrullination of GFAP may contribute to traumatic brain injury (Lazarus et al., 2015) and autoimmune disorders (Jin et al., 2013).

GFAP is encoded by a single gene on chromosome 17, expressed as 10 isoforms that differ with regard to splice sites (Brodie et al., 1998; Kamphuis et al., 2012; Moeton et al., 2016; Thomsen et al., 2013). The principle isoform, GFAP-a (432 amino acids),is highly expressed in central nervous system (CNS) glial cells and neurons, whereas isoforms R (beta), y (gamma), F (epsilon), x (kappa), and ((zeta) are expressed in many tissues and cell types beyond CNS neurons and glia (Kamphuis et al., 2012; Moeton et al., 2016; Thomsen et al., 2013).

Neuronal stress, caused by either disease or injury, evokes astrocyte activation as a response, including hypertrophy, proliferation, and increased GFAP expression (de Souza et al., 2020; Fan and He, 2016; Muccigrosso et al., 2016; Nawashiro et al., 1998). Initial post-injury glial activation is an acute response enabling recovery from brain insults (Dani et al., 2018; Donat et al., 2017). Long-term neuronal injury or stress causes chronic neuroinflammation, however, with negative impacts on brain function (Calabrese et al., 2018; Streit et al., 2004). AD is diagnosed as dementia accompanied or preceded by neuronal pathology comprising tau-specific tangles and amyloid plaques (Dani et al., 2018; Drummond et al., 2018; Hoenig et al., 2020). Elevated GFAP in hippocampi of patients with AD was observed, relative to age-matched controls (Ayyadevara et al., 2016b).

The Examples show the role of GFAP and its PTMs in protein aggregation. The Examples demonstrate hyperphosphorylation of GFAP in AD hippocampus, relative to age-matched control (AMC) GFAP that lacks substantial phosphorylation. AD-specific phosphorylations were attenuated by RNAi knockdowns of upstream kinases that may target the modified GFAP sites, each of which resulted in a marked reduction of amyloid deposition by human neuroblastoma and glioma cells in vitro. Approximately 750,000 small molecule structures from the ChemBridge library were screened to identify drug candidates with specific affinity for partially unfolded GFAP. One of these, termed MSR1 (FIG. 6), was particularly effective in reducing protein aggregation and pathology in a variety of AD models (human cells or C. elegans).

One aspect of the invention provides for the use of a compound that stably binds a Glial Fibrillary Acidic Protein (GFAP). One aspect of the invention is to provide a method for the reducing protein abundance in aggregates. The method comprises administering to a subject an effective amount of a compound that stably binds Glial Fibrillary Acidic Protein (GFAP). The term “protein aggregate” refers to an aggregate of protein and other constituents, formed in an intracellular or extracellular process by which misfolded or intrinsically disordered proteins adopt a conformation that causes their coalescence into insoluble aggregates, which may be globular or organized as fibrils. A protein aggregate may be insoluble in a moderately strong detergent, such as sarkosyl(sodium lauryl sarcosinate). “Aggregate protein” refers to a protein found or capable of being found in a protein aggregate.

The term “reducing aggregate protein abundance” refers to suppressing protein aggregation. In some embodiments, protein aggregation is suppressed by 50% or more. In some embodiments, protein aggregation is suppressed by 55%, 60%, 65%, 70%, 75%, or more. As demonstrated in the Examples, MSR1 suppressed protein aggregation by 60-75% in a variety of model systems.

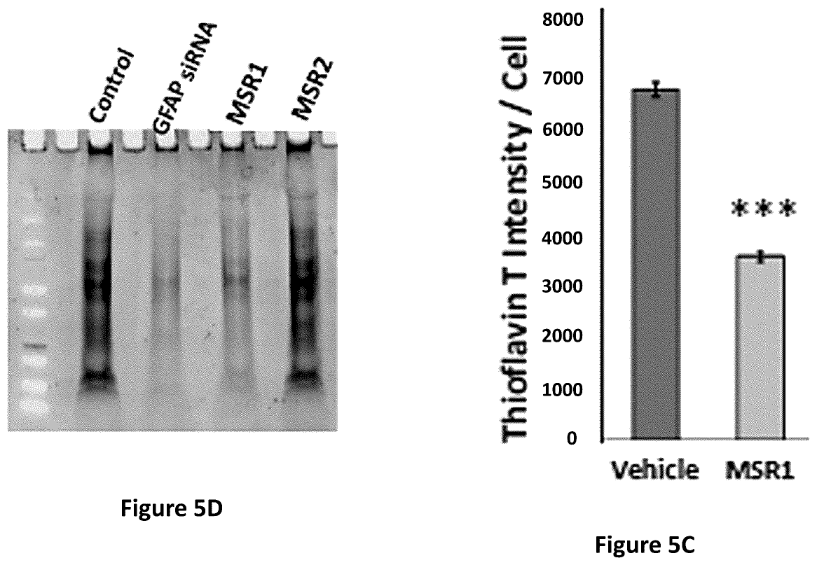

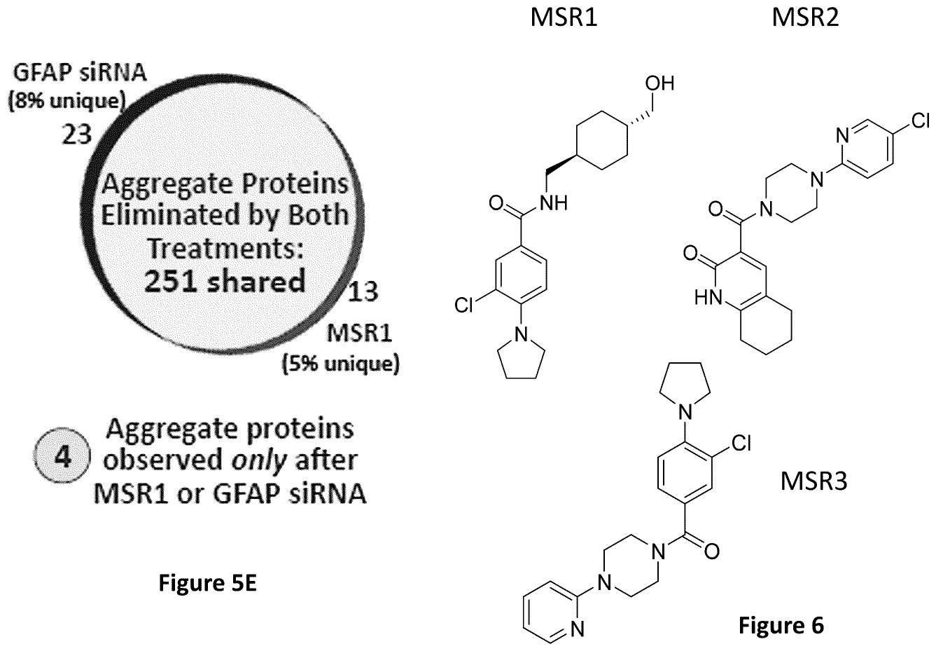

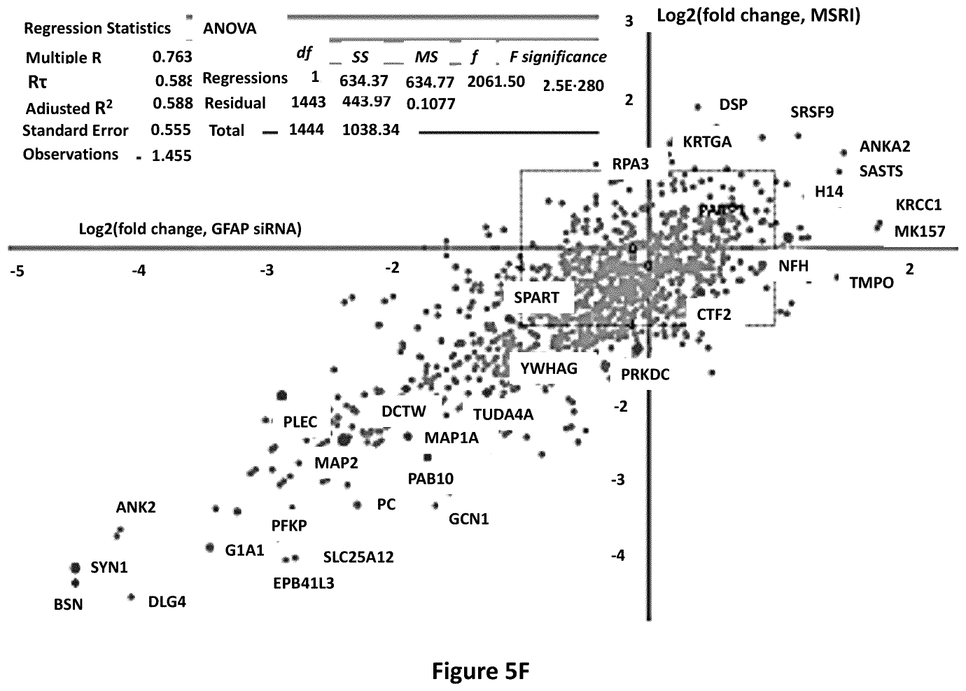

The abundance of a plurality of different aggregate proteins may be reduced. In some embodiments, 50, 100, 150, 200, or 250 or more aggregate proteins are partially or completely removed from a protein aggregate. Aggregate proteins that may be reduced include, without limitation those shown in FIG. 5F, such as BSN, SYN1, DLG4, ANK2, GJA1, EPB41L3, SLC25A12, PFKP, PC, MAP2, PLEC, GCN1, RAB10, MAP1A, DCTN, TUBA4A, YWHAG, PRKDC, SPART, EEF2 as well as others. In some embodiments, the aggregate protein may include immediate neighbors of GFAP such as BSN, SYN1, MAP2, PLEC, RAB10, MAP1A, DCTN, TUBA4A, SPART, PRKDC, EEF2, PARP1, NFH, H14, or any combination thereof. “Immediate neighbor” refers to adjacency within the aggregates. Other proteins may not directly contact GFAP but may still be “connected” to GFAP through a succession of other more GFAP-proximal proteins. Aggregate proteins that may be partially or completely removed from a protein aggregate may include RPS4X, SLC25A5, PSMC1, Cllorf98, PRPF40A, TRMT6, CCT5, LARP1, MSH6, UBA1, CSDE1, TARS, EIF2A, UTP20, SNRPE, UQCRC2, CYFIP2, ALDOA, PDS5A, AHCTF1, BANF1, YWHAQ, EP400, SMARCA4, IPO5, DLST, MRPL49, PPP1CB, SLC25A13, GPI, MYH9, GSTM3, RIF1, CHD2, ELAVL4, VARS, SNRPD2, DPYSL3, NSD2, SEPTIN2, PHOX2A, HSPB1, GNAI2, AGO2, FLNB, YWHAB, CHD8, IQGAP1, RBM8A, PDS5B, MTHFD1, MRPL38, EMD, GNAI1, EXOSC7, NPEPPS, CEP170, GAK, PGK1, USP10, XPO5, DPYSL5, LUC7L, FYN, PRMT1, PYGB, NOC3L, ATP2A2, LRPPRC, SF3B1, PDHB, LYN, PDHA1, STRAP, MARS, FLII, FSCN1, TUBB1, TUBB, TUBB3, CDK18, IARS, FLOT2, TUBB6, AGO1, WDR1, RANBP2, CBX1, IPO7, TLN1, NDUFS3, ATAD5, TUBB4B, YWHAG, CRMP1, GLUD1, TUBB2B, NOC2L, GTF3C3, HSPA4, DYNLL2, COX4I1, SEPTIN6, ATP5F1B, SNRPA, RAB39A, TUBB2A, HEATRI, DYNLL1, ZMYM4, CDK12, RAB1B, CHD7, SEPTIN1I, PRDX4, ARHGEF2, CLTC, GNA13, HCFC1, YES1, APEH, SIN3A, COX5A, COX5B, LARS, RPS23, AGO3, WDR33, RAB39B, YWHAH, DYNCIHI, PPP1CC, SEPTIN8, SPTBN1, PPP1CA, SART3, TUBB4A, CDK5, NAP1L1, RAB6A, SLC25A11, TUBAIB, SEPTIN7, EXOSC6, SOGA1, DDX19A, ATP13A1, MYO18A, CBR1, GNB2, TUBAL3, CAP1, MYO1B, CTNND1, PUF60, CWC22, MARK3, PLP1, USO1, TUBA4A, DPYSL4, ABCF2, CHD4, ALDOC, GCN1, DPYSL2, TUBA8, ZNF462, GDI1, HSPA4L, MBP, DLAT, FLOT1, PPIL1, HRNR, MAST3, YLPM1, MARK2, VDAC3, ACO2, KIF21A, GNAO1, NEFL, RAB10, INA, CAND1, SLC25A22, STXBP1, NEFM, GNB1, ATIC, AP1B1, AP2M1, CACNA2D1, NIPBL, MAP1B, RAC1, ACTN4, SUCLA2, ATP6AP1, DNAJB2, ALDH2, ATP2B3, CAMK2A, GOT2, DCTN1, AGAP3, MACF1, FARP1, DHX36, CD59, CDC42BPA, SPTAN1, AP2B1, DNM2, GFAP, CAMK2D, PLEC, ACTN1, ATP2B4, THY1, MAP1A, SPTBN2, CKMT1A, TUFM, MAP2, HSPA12A, ATP2B2, CNP, USP5, AP2A2, AARS, ATP1A1, NSF, CAMK2G, LONP1, AP2A1, CKB, ATP2B1, MYCBP2, WDR37, PFKL, EPB41L3, SLC25A12, PFKM, CNTN1, CAMK2B, SPTB, GLS, ATP6VOA1, ATP1A3, PFKP, DNM1, IARS2, ATP1A2, DNM3, DCLK1, PC, GJA1, DLG4, ANK2, CNTNAP1, BSN, SYN1, or any combination thereof.

Another aspect of the invention is to provide a method for the increasing an aggregate protein abundance in a protein aggregate. The method comprises administering to a subject an effective amount of a compound that stably binds Glial Fibrillary Acidic Protein (GFAP). The term “increase aggregate protein abundance” refers to suppressing protein aggregation. In some embodiments, protein aggregation is increased by 50% or more. In some embodiments, protein aggregation is increased by 55%, 60%, 65%, 70%, 75%, or more. The abundance of a plurality of different aggregate proteins may be increase. Aggregate proteins that may be increased include, without limitation, CBX8, TSPYL5, CDK2, KRT33B, or any combination thereof.

The term “stably bind” refers to a compound having a ΔG_{binding} to a protein of less than 0 kcal/mol (i.e., a negative energy). In some embodiments, the compound has a ΔG_{binding} of less than −40 kcal/mol. In some embodiments, the compound has a ΔG_{binding} of less than −43 kcal/mol, less than −46 kcal/mol, less than −49 kcal/mol, less than −50 kcal/mol, or less than −52 kcal/mol.

The term “ΔG_{binding}” refers to the change in Gibbs free energy of binding, which estimates the predicted affinity between a ligand and a protein associated with a binding process. The magnitude of the binding affinity is a measure of how strong the interaction is between the ligand and the protein, and hence it is often directly related to the potency of the ligand.

The term “negligible affinity” refers to a compound having a ΔG_{binding} to a protein of greater than or equal to −7 kcal/mol.

Exemplary compounds that stably bind GFAP include, without limitation: MSR1, MSR2, or MSR3.

The term “MSR1” refers to the compound 3-chloro-N-{[trans-4-(hydroxymethyl)cyclo-hexyl]methyl}-4-pyrrolidin-1-ylbenzamide. It has a molecular formula of C₁₉H₂₇ClN₂O₂. The structure of MSR1 is shown in FIG. 6.

The term “MSR2” refers to the compound 3-{[4-(5-chloro-2-pyridinyl)-1-piperazinyl]carbonyl}-5,6,7,8-tetrahydro-2(1H)-quinolinone. It has a molecular formula of C₁₉H₂₁ClN₄O₂. The structure of MSR2 is shown in FIG. 6.

The term “MSR3” refers to the compound 1-(3-chloro-4-pyrrolidin-1-ylbenzoyl)-4-pyridin-2-ylpiperazine. It has a molecular formula of C₂₀H₂₃ClN₄O. The structure of MSR3 is shown in FIG. 6.

In some embodiments, the compound has negligible affinity for α-tubulin, β-tubulin, or an oligomer thereof. The terms “α-tubulin“and” β-tubulin” refer to the α- and β-subunits of the protein tubulin. Tubulin is the protein that polymerizes into long chains or filaments that form microtubules, hollow fibers which serve as an intracellular skeletal system for living cells. Microtubules have the ability to shift through various conformations, thereby enabling a cell to undergo mitosis or to conduct intracellular transport. α-tubulin is a fairly ubiquitous microtubule component, whereas β-tubulin is specific to neuronal microtubules. α- and β-tubulins both co-polymerize into microtubules, a major component of the eukaryotic cytoskeleton.

The terms “oligomer” and “polymer” refer to proteins composed of more than one subunit (polypeptide chain). As such, oligomeric proteins possess a quaternary structure, generally considered to be the highest level of organization within the protein structural hierarchy.

Oligomeric proteins may be composed either exclusively of several copies of identical polypeptide chains, in which case they are termed homo-oligomers, or alternatively by at least one copy of two or more distinct classes of polypeptide chains (hetero-oligomers).

The compounds that stably bind GFAP used in the methods disclosed herein may be formulated as pharmaceutical compositions that include: (a) a therapeutically effective amount of one or more protein degraders as described herein and (b) one or more pharmaceutically acceptable carriers, excipients, or diluents. The pharmaceutical composition may include the compound in a range of about 0.1 to 5000 mg (preferably about 0.5 to 500 mg, and more preferably about 1 to 100 mg). The pharmaceutical composition may be administered to provide the compound at a daily dose of about 0.1 to 500 mg/kg body weight (preferably about 0.5 to 20 mg/kg body weight, more preferably about 0.1 to 10 mg/kg body weight). In some embodiments, the pharmaceutical composition may further comprise a bioactive agent.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended-release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers, such as microcrystalline cellulose, lactose, dibasic calcium phosphate, saccharides, and mixtures of any of the foregoing.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Alternatively, only the sodium bicarbonate component of the effervescent couple may be present.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The compounds utilized in the methods disclosed herein may be administered in conventional dosage forms prepared by combining the active ingredient with standard pharmaceutical carriers or diluents according to conventional procedures well known in the art.

These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route, intraperitoneal injection, and topically, such as via eye drop. Such formulations may be prepared by any method known in the art of pharmacy, for example by bringing into association the active ingredient with the carrier(s) or excipient(s).

The formulations may be presented in unit-dose or multi-dose containers.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures. The compounds for use according to the methods of disclosed herein may be administered as a single compound or a combination of compounds. For example, a compound may be administered as a single compound or in combination with another compound that has the same or different pharmacological activity.

As indicated above, pharmaceutically acceptable salts of the compounds are contemplated and also may be utilized in the disclosed methods. The term “pharmaceutically acceptable salt” as used herein, refers to salts of the compounds that are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

The particular counter-ion forming a part of any salt of a compound disclosed herein is may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

An aspect of the technology provides for a method for treating of subject in need of a compound that stably bind GFAP. Suitably, method may comprise administering to the subject an effective amount of a compound that stably binds GFAP. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein, a “subject” may be interchangeable with “patient” or “individual” and means an animal, which may be a human or non-human animal, in need of treatment. A “subject in need of treatment” may include a subject having a disease, disorder, or condition that is responsive to therapy with the compounds disclosed herein alone or in combination with another bioactive agent. Examples of disease, disorders, or conditions include, but are not limited to, neurodegenerative diseases, such as Alzheimer's disease or Parkinson's disease, traumatic brain injury, other neurological conditions, or cardiovascular conditions. The term “bioactive agent” is used to describe an agent, other than a conjugate, which is used in combination with the compound as an agent with biological activity to assist in effecting an intended therapy, inhibition and/or prevention/prophylaxis for which the present compounds are used.

As used herein the term “effective amount” refers to the amount or dose of the compound, such as upon single or multiple dose administration to the subject, which provides the desired effect. For example, with respect to the treatment of a cancer, an effective amount will refer to the amount of a therapeutic agent that decreases the rate of tumor growth, decreases tumor mass, decreases the number of metastases, increases time to tumor progression, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. With respect to sensitization, an effective amount will refer to the amount of a therapeutic agent that results in sensitization of the subject or cell as described above.

An effective amount can be determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

In some embodiments, the subject has a neurodegenerative disorder. The term “neurodegenerative disorder” refers to a type of disorder in which nerve cells of the brain or peripheral nervous system lose function over time and ultimately die. Neurodegenerative disorders are characterized by a collapse in proteostasis, shown by the accumulation of insoluble protein aggregates in the brain. Common neurodegenerative disorders include, but are not limited to, Alzheimer's disease, Parkinson's disease, prion disease, motor neurone disease, Huntington's disease, spinocerebellar ataxia, spinal muscular atrophy, amyotrophic lateral sclerosis, Friedreich ataxia, Lewy body disease, multiple system atrophy, progressive supranuclear palsy, etc.

In some embodiments, the subject has Alzheimer's disease (AD). The term “Alzheimer's disease” refers to a progressive neurologic disorder that causes the brain to shrink (atrophy) and brain cells to die. AD is the most common cause of dementia and continuous decline in thinking, behavioral and social skills. Brain cell connections and the cells themselves degenerate and die in AD patients, eventually destroying memory and other important mental functions.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

### Methods

C. elegans strains: Nematode strains used in this study were all obtained from the Caenorhabditis Genetics Center (CGC; Minneapolis, MN). Nematode models of Alzheimer's disease-like arnyloidosis were CL4176 [smg-1^{ts}; myo-3p::Aβ₁₋₄₂::let-851 3′-UTR; rol-6(sul006)] expressing human Aβ₁₋₄₂ in muscle; and CL2355 [smg-l^{ts}; snb-1::Aβ1-42::3′UTR(long); mtl-2::gfp] expressing human Aβ₁₋₄₂ in all neurons. Strain AM141 expresses a polyglutamine reporter (Q40::YFP) in muscle, and thus serves as a model of glutamine-tract aggregation similar to that observed in Huntington disease and several other neuropathologies (Morley et al., 2002). All strains were propagated at 20° C. on2% (w/v) agar plates containing nematode growth medium (NGM), overlaid with E col strain OP50 unless otherwise noted.

Chemotaxis and paralysis assay)s in Aβ-tragsgenic C elegans strains CL2355 and CL4176. Transgenic C. elegans strains, expressing Aβ₁₋₄₂ in neurons (C2355) or in muscle (CL4176), were grown at 20° C. with ample 1. coli (OP50) bacteria, and on day 1 of adult life worms were lysed to release eggs and thus generate a synchronized cohort. Eggs were then placed on I00-mm NGM-agar Petri dishes seeded with RNAi-expressing bacteria (E coil H-T1′15) targeting a GFAP orthologue, or empty-vector control bacteria. Worms at the L3-L4 transition were upshifted to 25.5° C. to induce human Aβ₁₋₄₂ transgene expression and assayed after 48 hours. Chemotaxis (Dosanjh et al., 2010) and paralysis (Dostal and Link, 2010) assays were performed as described previously (Ayyadevara et al., 2017; Ayyadevara et al., 2016b; Ayyadevara et al., 2016d; Kakraba et al., 2019).

Paralysis assays in human tau-expressing C. elegans strain VH255. Transgenic C. elegans strain VH255, with pan-neuronal expression of human tau (Brandt et al., 2009), are maintained at 25° C. on agar plates overlaid with a lawn of E. coli (OP50) bacteria. Worms are lysed in alkaline hypochlorite solution to obtain unlaid eggs used to generate a synchronized culture. The eggs were then transferred to 100-mm NGM-agar plates, to which MSR1 or MSR2 had been added to a final concentration of 1 μM. For ifp-1 RNAi knockdown, the bacterial lawn consisted of strain HT115 expressing an exonic segment of double-stranded ifp-I RNA (see next section). The worms were washed and added to fresh, drug-equilibrated plates every two days. Assays were performed on day-3 adults to assess the fraction of paralyzed worms.

RNAi in C.elegans. RNA-mediated interference (RNAi) is achieved by feeding bacteria that express double-stranded RNA corresponding to an exonic fragment of the mRNA target (Ayyadevara et al., 2016c; Fire et al., 1991; Fire et al., 1998; Kakraba et al., 2019). Briefly, worm cultures were synchronized by alkaline hypochlorite lysis to release unlaid eggs. Eggs prior to hatch, or late-L4 larvae, were placed onto IPTG-containing NGM plates seeded with bacteria (E. coli HT115[DE3]) carrying the empty vector L4440 (pPD129.36) or bacterial clones expressing ifp-1 (homologous to human GFAP), let-502 (orthologous to human ROCK1), akt-2 (AKT2), kin-1 (PKA) or grk-1 (BARK). Worms on adult day 3 (5.5 days post-hatch) were imaged to assess total aggregate fluorescence (strain AM141), or were assessed for paralysis (VH255) or chemotaxis toward n-butanol (CL2355).

siRNA knockdowns and Thioflavin-T staining of human cells. SH-SY5Y-APP_{Sw} cells from an exponentially growing culture were trypsinized, rinsed, plated at 10,000 cells per well in 96-well plates, and grown 16 h at 37° C. in “DMEM+F12” (Life Technologies) supplemented with 10% fetal bovine serum (FBS). Cells at ˜40% confluence were transfected with short interfering RNA (siRNA) constructs targeting GFAP (SAS1_Hs01 00227618), AKT2 (SAS1_Hs01 00035058), ROCK1 (SASi Hs 00065571), BARK (SASi Hs 00039321), or PKA (SAS1 Hs 00217223), all obtained from Millipore-Sigma (St. Louis, USA). Transfections with the indicated siRNAs were performed using RNAiMax reagent (Life Technologies) according to the manufacturer's directions. Cells at 48 hours post-transfection were fixed in 4% v/v formaldehyde and stained in a dark container with 0.1% w/v Thioflavin T. After 4 washes in PBS, cells were covered with Antifade+DAPI (EMD-Millipore) and fluorescence was captured in green and blue channels, using a Keyence fluorescence microscope with motorized stage for automated well-by-well imaging, 9 fields per well. Thioflavin-T fluorescence intensity was divided by the number of DAPI+nuclei in each well, yielding ratios of amyloid per cell, summarized as mean±SD.

MSR1 treatment of SH-SY5Y-APP_{Sw}, cells. Human neuroblastoma (SH-SY5Y-APP_{Sw}) cells were grown as previously described (Kakraba et al., 2)19, Liu et al., 2005). SH-SY5Y-APP_{Sw} cells, expressing an aggregation-prone “Swedish” double mutant of amyloid precursor protein (APP_{Sw}), were grown in DMEM plus 10% (v/v) FBS at 37° C. Cells were suspended in trypsin/EDTA and rinsed in buffer prior to replating or harvesting. Immediately preceding assay, cells were grown 48 hr in the presence of 10-μM MSR1 dissolved in DMSO (0.02% final concentration), or 0.02% DMSO (solvent alone) for control cells. Cells were harvested, total protein was isolated, and aggregate proteins were purified as described below.

Western-blotting analysis of glial (T986) cells for pGFAP and ROCK1. Human glioblastoma cells (T98G) were maintained in Dulbecco's modified Eagle's medium (DMEM; Invitrogen/Life Technologies, Grand Island, NY), supplemented to 10% FBS, v/v. Cells were harvested and their proteins extracted in lysis buffer (50-mM Tris-HCl, pH 7.5, 150-mM NaCl, 1% w/v Nonidet P40, 0.1% SDS, 0.5% sodium deoxycholate) and quantified with Bradford reagent (Bio-Rad). Protein aliquots (50 g) were electrophoresed 2 hr at 100 V on a 4-20% gradient bis-tris acrylamide gel (BioRad Life Science, Hercules, CA), and transferred to nitrocellulose membranes. After pre-incubation with BSA blocker (Pierce), blot membranes were probed with rabbit antibody to pGFAP or ROCK-1 (Cell Signaling, 1:100 dilution) overnight at 4° C. After washes, membranes were incubated 1 hour at room temperature with a secondary antibody: either HRP-conjugated goat anti-rabbit IgG (AbCam, 1:10,000 dilution) or rabbit anti-goat IgG (Rockland Immuno-chemicals, Gilbertsville, PA), and developed using an ECL chemiluminescence detection kit (Pierce). Data were digitized, and analyzed using ImageJ software (NIH).

Isolation of aggregate proteins. Cultured human cells were collected, flash frozen in liquid nitrogen, and homogenized at 0° C. in the presence of buffer containing nonionic detergent (1% v/v NP40, 20-mM Hepes pH 7.4, 300-mM NaCl, 2-mM MgCl₂, and protease/phosphatase inhibitors [CalBiochem]) [11, 12, 37]. Lysates were centrifuged (5 min, 3000 rpm at 4° C.) to remove debris. After removal of cytosolic proteins (soluble in 1% NP40 nonionic detergent) by centrifugation (18 min, 13,000×g at 4° C.), protein pellets were brought to pH 7.4 with 0.1-M HEPES buffer containing 1% v/v sarkosyl(sodium lauryl sarcosinate) and 5-mM EDTA, and centrifuged 30 min at 100,000×g. Pellet proteins (the sarkosyl-insoluble fraction) were resuspended in Laemmli loading buffer (containing 50-mM dithiothreitol and 2% v/v SDS, sodium dodecyl sulfate), heated 5 min at 95° C. to dissolve proteins soluble in this buffer, and separated by electrophoresis on 4-20% polyacrylamide gradient gels containing 1% v/v SDS. Gels were stained with SYPRO Ruby (ThermoFisher) or Coomassie Blue to visualize protein. Gel lanes were robotically cut into 1-mm slices, and digested thoroughly with trypsin. Proteins in each slice were identified by mass spectrometry as described previously (Ayyadevara et al., 2015; Ayyadevara et al., 2016a; Ayyadevara et al., 2016b; Ayyadevara et al., 2016c; Ayyadevara et al., 2016d; Balasubramaniam et al., 2018).

Modeling and MD simulation of GFAP structure. The three-dimensional structure of GFAP was modelled using fold-recognition and ab initio structure prediction methods implemented by I-TASSER server-based algorithms. I-TASSER generates 5 different models, of which the lowest-energy conformer was chosen for further processing. For Molecular Dynamic (MD) simulations, we used the protein-preparation wizard within the Schrodinger Desmond simulation suite to prepare modelled structures. Physiological conditions were approximated during simulations by creating an orthorhombic simulation box filled with Simple Point Charge (SPC) water, neutralization of locally charged sites with appropriate counterions (Na⁺, Cl⁻), and further addition of 0.15 M NaCl to achieve a physiological isotonic state. For equilibration, temperature and pressure are held at 300° K and 1.1023 bar, respectively. The random sampling seed input is changed for each run, and each 200- to 500-ns simulation is repeated at least three times. Simulations with phosphorylations were incorporated using the Maestro “Mutate residue” plug-in, in which specified residues were converted to their phosphorylated form. Trajectories were visualized with VMD, and analyzed using BIOVIA Discovery Studio (Dassault Systemes).

Virtual screening of a target protein against molecular-structure libraries. High-throughput virtual screening of the ChemBridge molecular-structure library for docking to GFAP was initially conducted using the Schrodinger Suite Glide module. ChemBridge structures were retrieved in 2D format and prepared using LigPrep wizard (Schrodinger Suite). A 3-stage strategy was employed to improve the efficiency of virtual drug screening: (i.) the entire library of ˜750,000 molecular structures was virtually docked to the GFAP protein in Glide's high-throughput mode; (ii.) the top 1% of structures from high-throughput screening were then docked again to GFAP in Glide's standard-precision mode; and (iii.) for the top 1% of structures from standard-precision docking, binding free energies were predicted under MM-GBSA conditions using the Schrodinger Suite Prime module. Protein-ligand complexes with highest avidity (lowest ΔG_{binding}) were simulated using the Schrodinger Desmond module to assess their stability overtime.

Statistical analyses. For replicated assays of protein aggregation, chemotaxis, and paralysis, differences between control and experimental groups were assessed for significance by the Fisher-Behrens heteroscedastic t-test (appropriate to samples of unequal or unknown variance), treating each experiment as a single point. Within experiments, differences in proportions (fractional paralysis or chemotaxis) were evaluated by chi-squared or Fisher exact tests, as appropriate based on sample counts.

### Results

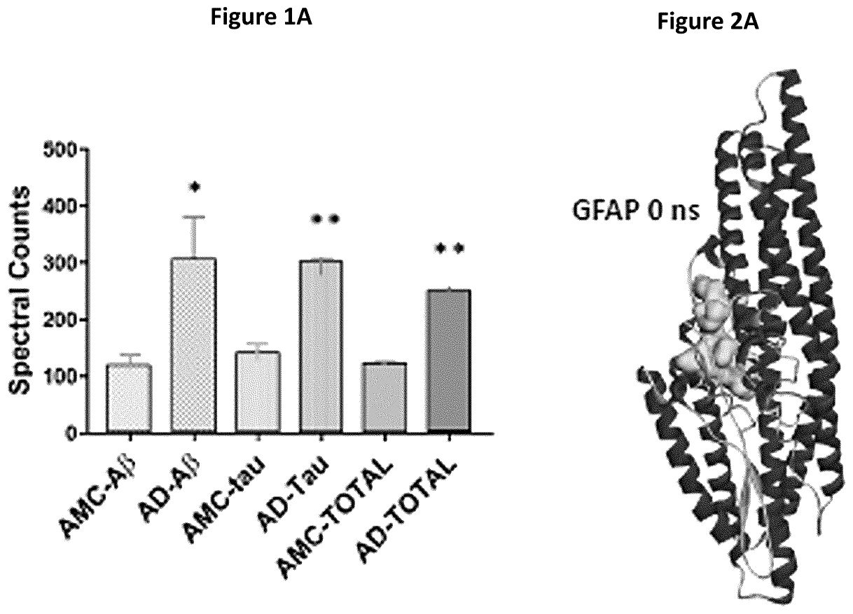

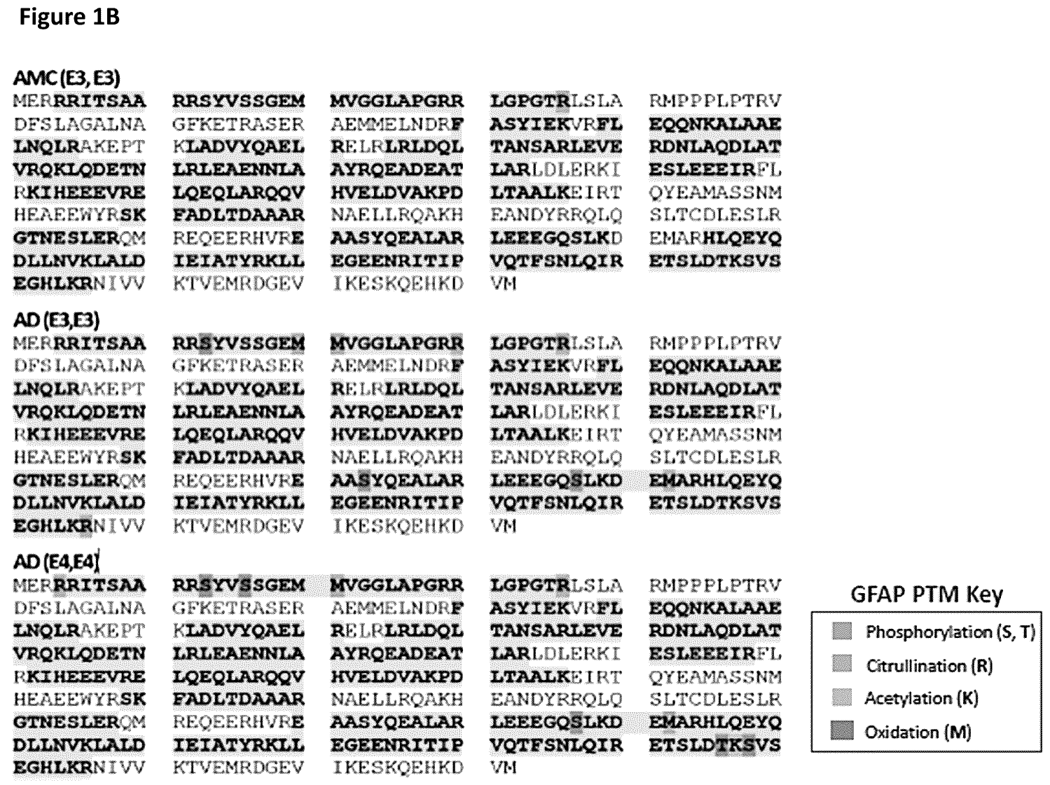

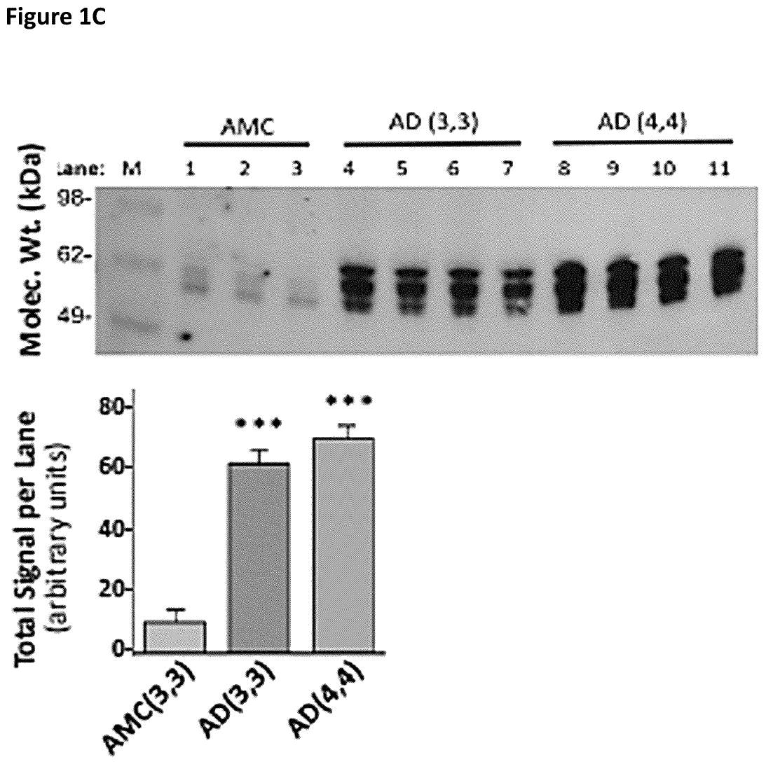

Glial fibrillary acidic protein is enriched, hyperphosphorylated, and oxidized in aggregates formed in Alzheimer's hippocampus. Glial fibrillary acidic protein (GFAP), a largely unstructured protein, is enriched 2- to 2.5-fold in three subclasses of detergent-insoluble aggregates from AD hippocampus relative to age-matched control (AMC) hippocampus (FIG. 1A). Although GFAP in control aggregates has no prevalent post-translational modifications (PTMs), GFAP in AD aggregates is phosphorylated at 3-5 serine or threonine residues (FIG. 1i). Remarkably, the GFAP phosphorylation signature differed reproducibly between individuals carrying ApoE alleles F3,F3 or F4,F4 (abbreviated “3,3” and “4,4” respectively). Western blots of AD(3,3) and AD(4,4) samples confirmed significant enrichment of hyperphosphorylated GFAP (hP-GFAP) in AD tissue relative to AMC (FIG. 1C); each genotype group differs from AMC(3,3) control samples at P≤0.0001.

PEAKS software (PTM module) was used to screen several other PTMs, but none were useful in distinguishing AD from AMC. In all groups, methylated arginine and lysine were observed at R88 and K95, whereas K107 is dimethylated, all >90%. Deamidation was not observed in >10% of spectral counts for any GFAP peptide, and pyroglutamine never exceeded 25% (data not shown).

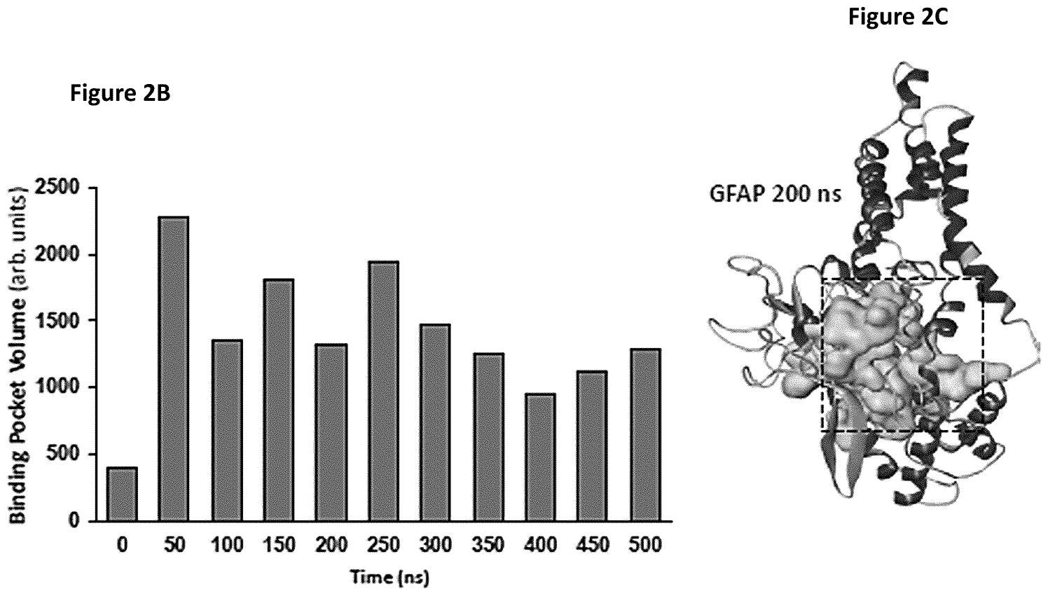

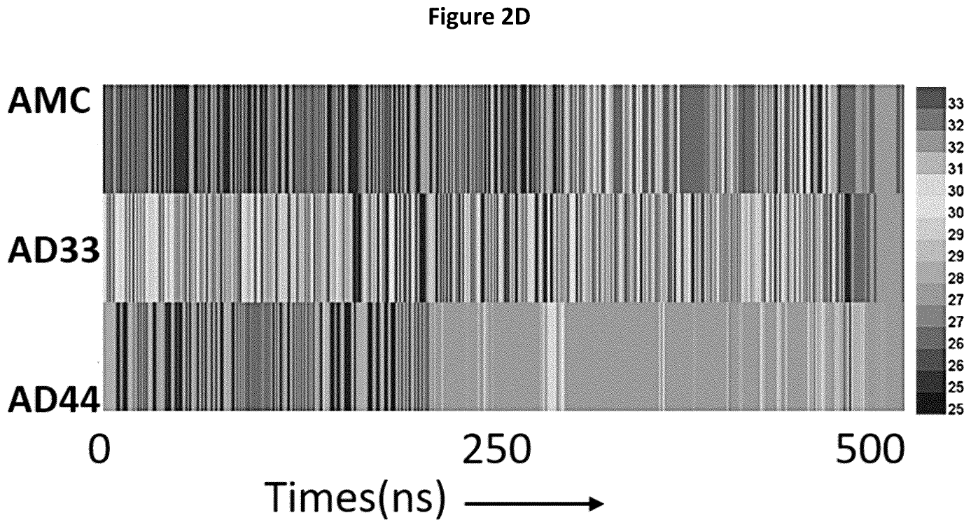

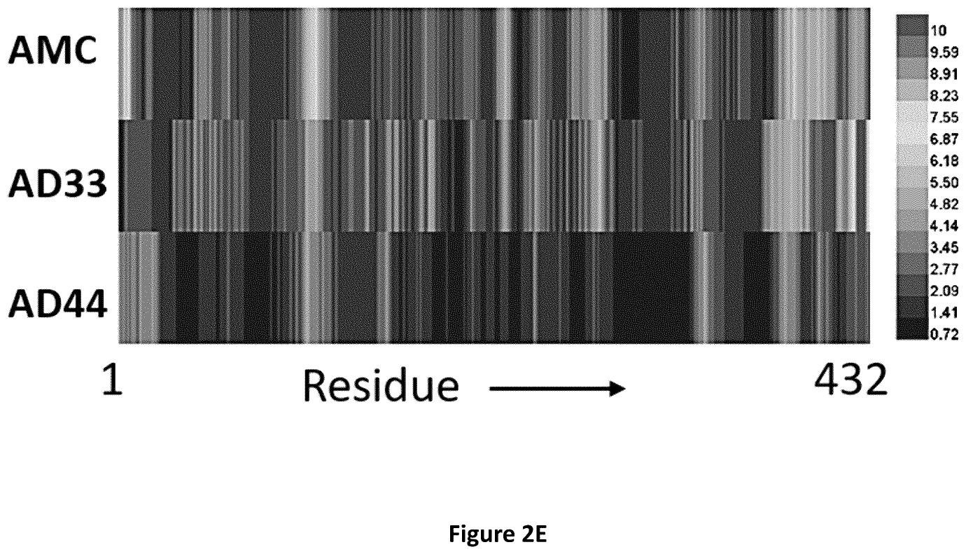

Molecular-dynamic simulations predict GFAP unfolding and identify a druggable pocket. Molecular-dynamic simulations of the hP-GFAP structures observed in AD aggregates (rendered by phosphomimetic substitutions) predict a more malleable GFAP structure in ApoE(3,3) individuals, but relatively greater structural rigidity in AD(4,4), compared to unphosphorylated GFAP as seen in AMC aggregates (FIGS. 2A-2E). Since GFAP is a largely disordered protein, its full-length structure has not been experimentally determined. We therefore predicted its three-dimensional structure using fold-recognition and ab initio procedures in I-TASSER (Yang et al., 2015). The resulting hypothetical structure comprises helices and loops (FIG. 2A), forming a small pocket or cavity near the inner groove of the protein.

In view of its disordered nature, the predicted protein structure is expected to be unstable and to unfold spontaneously, altering the orientation and drug-accessibility of the pocket. To explore the protein-unfolding trajectory, we simulated the predicted structure of fully-solvated GFAP for 0.5 s (500 ns). The volume of the druggable pocket (FIG. 2B), and several measures of atomic positional variation (see below), provide useful descriptors of structural change. These analyses support the anticipated unfolding of the initial GFAP structure, wherein the druggable pocket (FIGS. 2A and 2C) expands during the course of the simulation. We selected an intermediate, meta-stable structure (at 200 ns of simulation; FIG. 2C) to screen for small-molecule binding.

The observed differential phosphorylation of GFAP in AD aggregates (FIGS. 1A-1C) may be expected to impact GFAP structural dynamics. To evaluate this possibility, we computationally “mutated” the observed phosphorylated sites to glutamic acid (phospho-mimetic substitutions) and simulated the resulting structures for 0.5 s. Results support the expectation that GFAP hyperphosphorylation in the AD brain is likely to alter its structural stability. Root Mean Square Deviation (RMSD) of the GFAP atomic coordinates over this time interval is consistently lower for the unmodified molecule, “AMC(3,3) GFAP”, than for “AD(3,3) GFAP”, a phosphomimetic structure similar to that observed in AD(3,3) aggregates (FIG. 2D). “AD(4,4) GFAP” (a phosphomimic of GFAP observed in AD(4,4) aggregates) is initially a little more variable than AMC(3,3) GFAP but achieves a relatively stable structure that is maintained from −235 ns onward (FIG. 2D).

We also plotted the average Root Mean Square Fluctuation (RMSF) of individual residues over time, which indicates moderate-to-high positional fluctuation across the GFAP molecule, for both AMC(3,3) and AD(3,3) structures (FIG. 2E), in contrast to the relative rigidity of the AD(4,4) phosphomimetic structure. Together, these data (FIGS. 2D and 2E) support the prediction that AD-associated differential phosphorylations can modify GFAP structure, somewhat unstably in ApoE(3,3) but creating a relatively invariant conformation in ApoE(4,4).

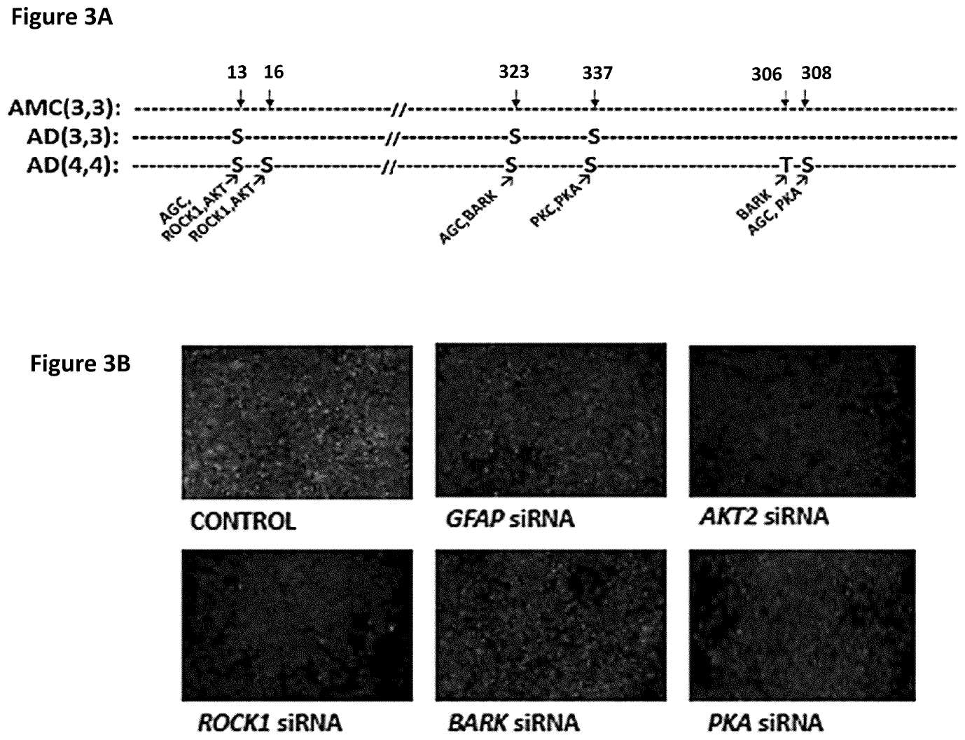

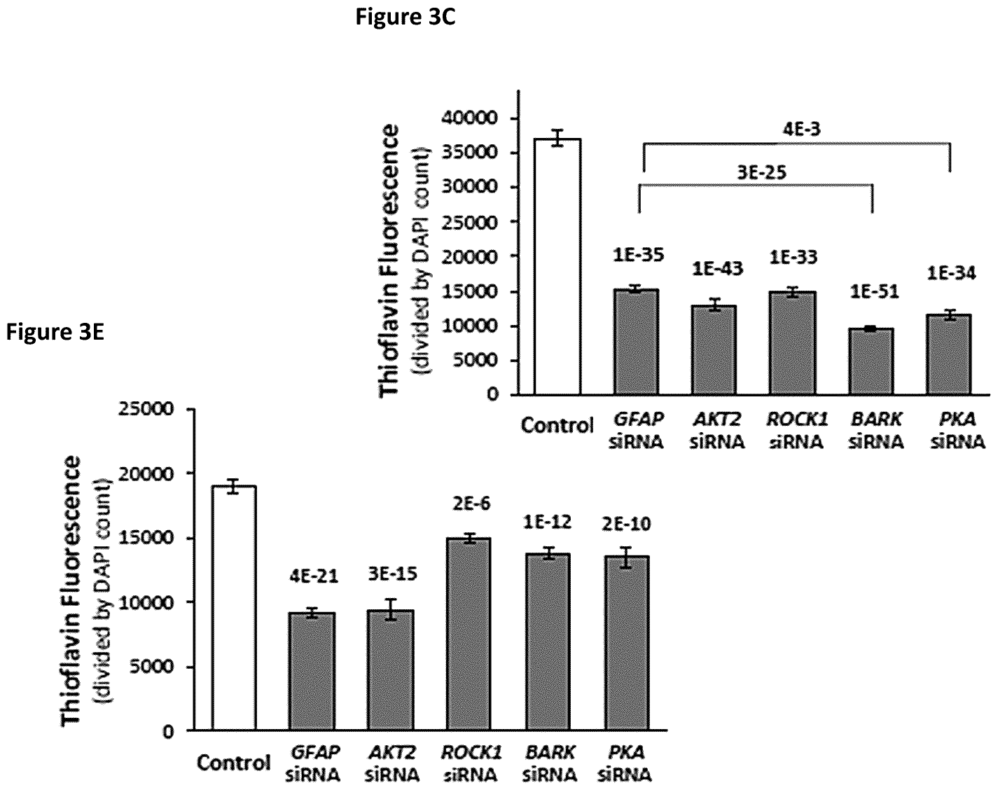

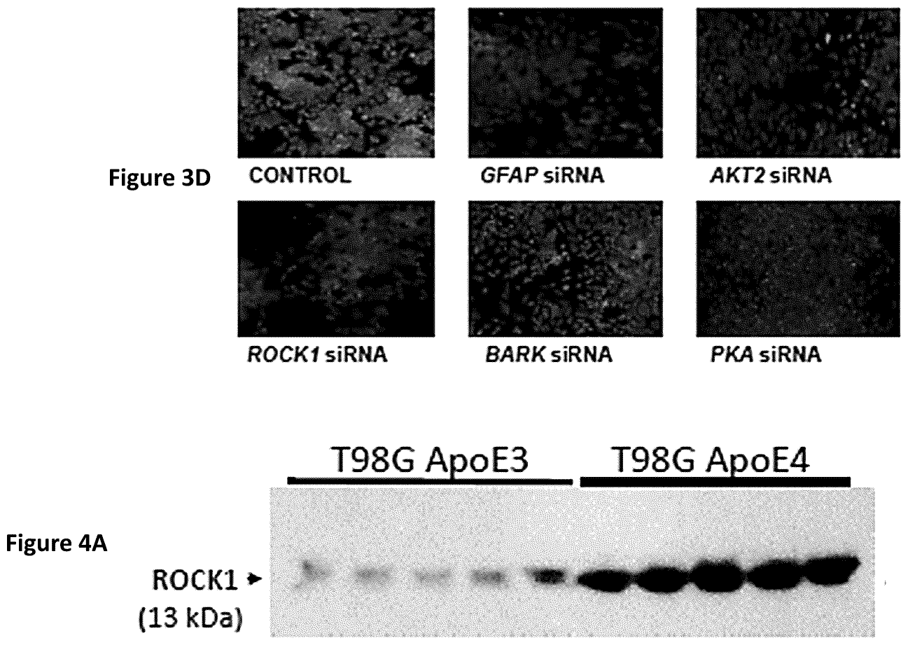

Identification of potential kinases mediating GFAP phosphorylation. Using phosphorylation-prediction software, NetPhos (http://www.cbs.dtu.dk/services/NetPhos) and GPS (http://gps.biocuckoo.org/online.php), we predicted the upstream kinases for each putative GFAP target residue (Table 1 and FIG. 3A). All of the implicated kinases (AKT2, ROCK1, BARK/GRK, and PKA), predicted to phosphorylate GFAP at the modified sites, had been previously implicated in AD pathogenesis or progression (Banerjee et al., 2021; Henderson et al., 2016; Ko et al., 2019; Obrenovich et al., 2009a; Obrenovich et al., 2009b; Obrenovich et al., 2006; Russo, 2019; Taylor et al., 2021; Zhang et al., 2020). We therefore tested the effects of individual kinase knockdowns on protein aggregation in human neuroblastoma cells (SH-SY5Y-APP_{Sw}; FIG. 3B, 3C) and human glioblastoma cells (T98G; FIG. 3D, 3E), with or without siRNA-mediated knockdown. KD of each kinase gene reduced aggregates in SH-SYSY-APP_{Sw} cells by 6070, similar to (or exceeding) the effect of GFAP siRNA (FIG. 3C). In T98G cells, only AKT2 siRNA relieved aggregation as effectively as GFAP siRNA (FIG. 3E).

TABLE 1
Kinases predicted to be capable of phosphorylating GFAP sites.

| PTM | Phosphorylated | | Peptide | Actual | Cutoff | SEQ ID |
|---|---|---|---|---|---|---|
| Position | Amino Acid | Kinase | Sequence | Score | Score | NO: |
| 13 | S | AGC, | ITSAARRSYVSSGEM | 27.88 | 20.90 | 1 |
| | | ROCK1, or | | | | |
| | | AKT/AKT2 | | | | |
| 16 | S | ROCK1 or | AARRSYVSSGEMMVG | 12.02 | 11.64 | 2 |
| | | AKT | | | | |
| 323 | S | AGC or | RHVREAASYQEALAR | 2.12 | 2.10 | 3 |
| | | BARK/GRK | | | | |
| 337 | S | PKA or | RLEEEGQSLKDEMAR | 119.80 | 104.65 | 4 |
| | | PKCa | | | | |
| 396 | T | BARK/GRK | IRETSLDTKSVSEGH | 171.89 | 151.15 | 5 |
| 398 | S | AGC or | ETSLDTKSVSEGHLK | 21.30 | 21.27 | 6 |
| | | PKA | | | | |

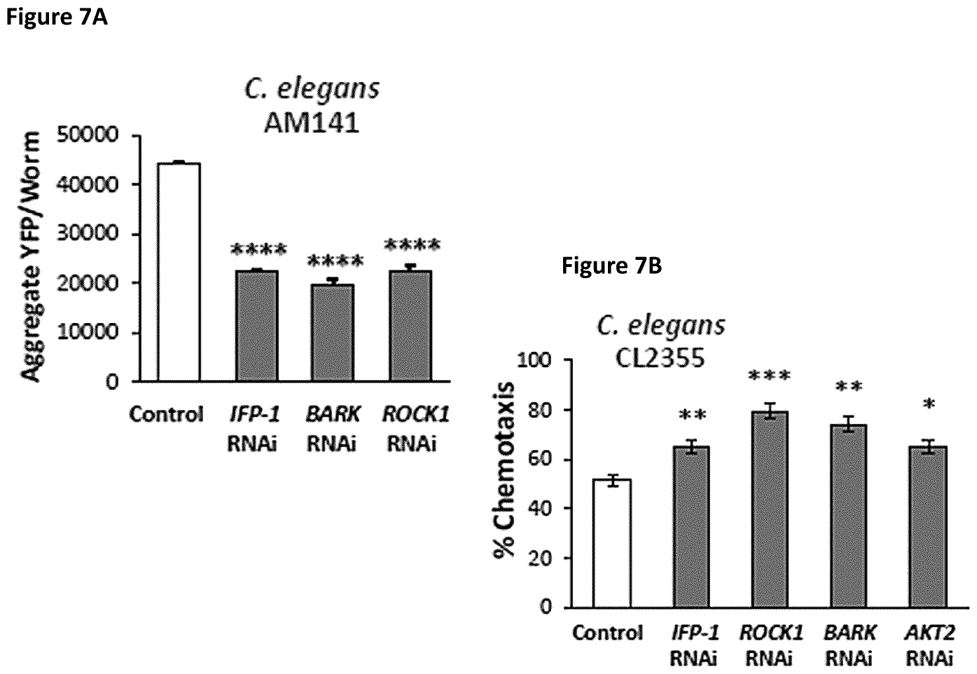

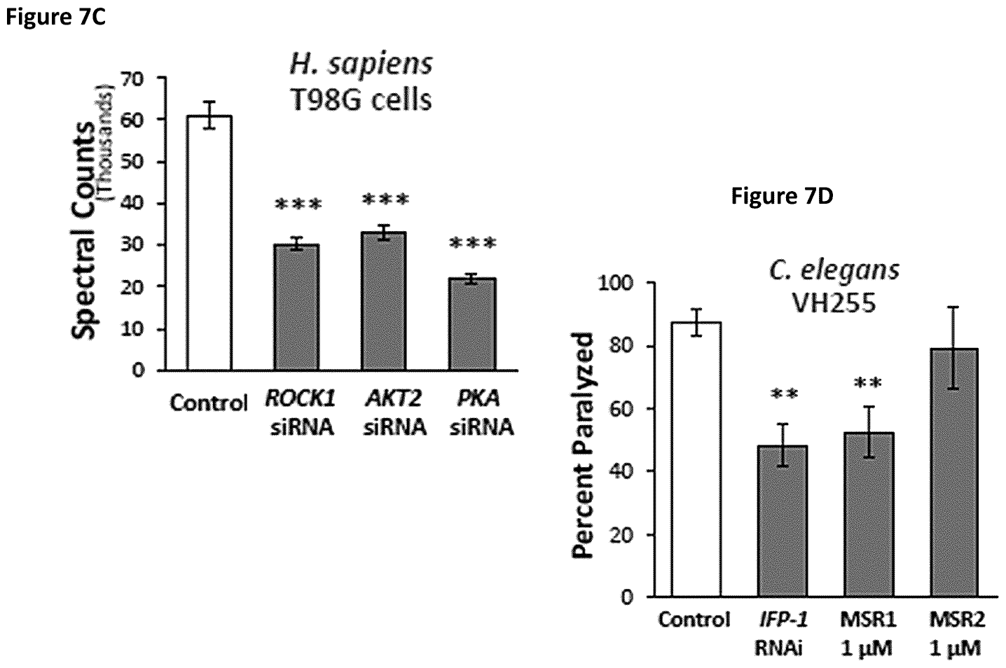

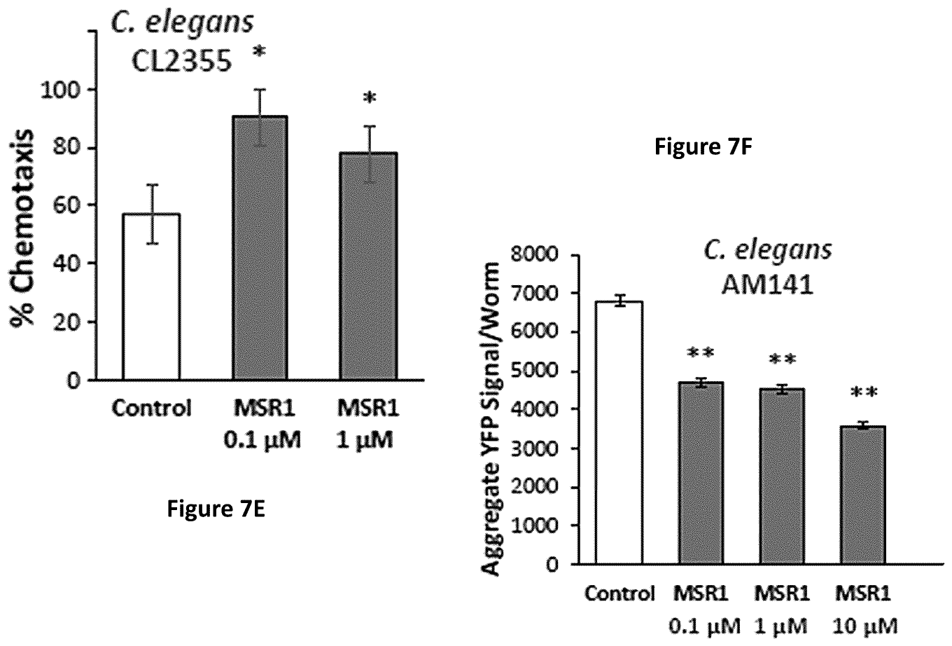

We also compared the impact of kinase-targeted knockdowns in C. elegans models of protein aggregation, using RNAi constructs to silence the closest nematode orthologs of these human kinases. We used aggregation models that simulate Huntington's disease via polyglutamine-array aggregation (strain AiM141), and Alzheimer's disease using neuronal expression of Aβ₁₋₄₂ peptide to form amyloid (CL2355), or muscle expression of human tau to form toxic aggregates that lead to paralysis (VH1255). In the Huntington model, total aggregate intensity per worm (FIG. 7A), i.e. the product of aggregate count per worm and mean YFP fluorescence per aggregate, was reduced 50-60% by knockdown of C. elegans genes orthologous to BARK/GRK or 15 ROCK1 (each P≤0.00005) similar to the effect of RNAi against ifp-1 (a partial homolog of GFAP). In a C. elegans model of AD-like neuronal amyloidosis (CL2355), chemotaxis declined after neuronal induction of an A3 transgene, causing fewer worms to migrate toward n-butanol (chemo-attractant). RNAi knockdowns of ROCK1, AKT2, or BARK GRK orthologs (let- 502, akt-2, and grk-2, respectively) rescued the chemotaxis defect at least as well asKD of ifp-1, an intermediate filament protein with homology to human GFP (FIG. 7B). These C. elegans results are similar to those observed in the human glioblastoma cell line, T98G, in which siRNA KD of ROCK1, AKT2, or PKA reduced total aggregate protein by 50-60% (FIG. 7C).

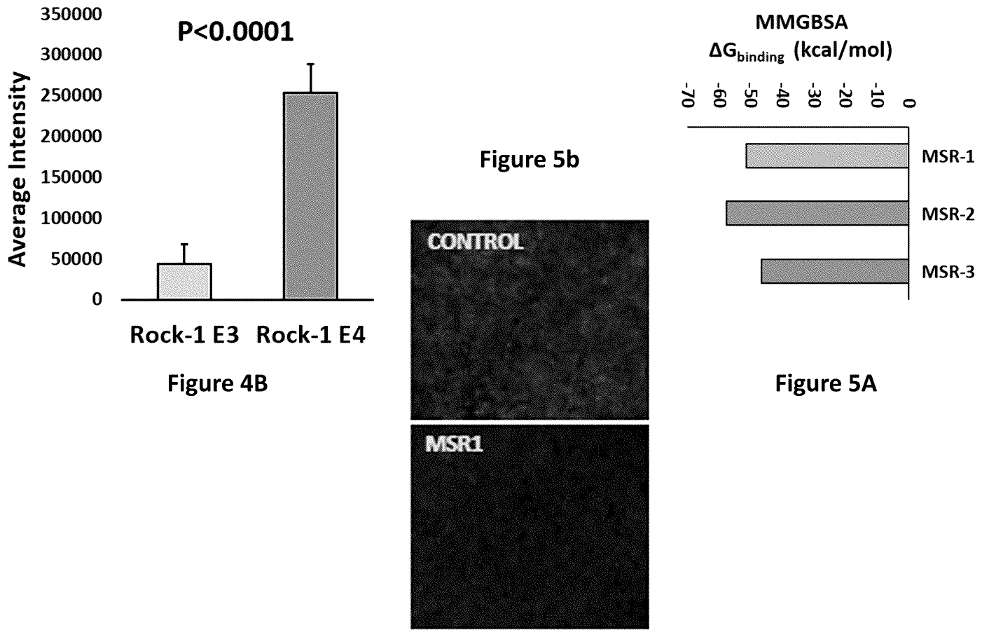

ROCK1 is increased in ApoE-expressing glioblastoma cells. Rho-associated protein kinase 1 (ROCK1) protein levels are elevated in mild cognitive impairment and AD, and its reduction by hemizygous knockout blunted the high amyloid levels seen in a mouse model of AD (Henderson et al., 2016). Since we observed a significant benefit from reducing ROCK1 levels both in human cells in culture and in intact C. elegans aggregation models, we measured its levels in T98G glioblastoma cells that overexpress either the APOE3 or APOE4 allele. ROCK1 protein levels were at least 6-fold higher in human glial cells that overexpress the APOE4 allele, than in the same cells expressing an APOE3 transgene (P≤0.0001; FIG. 4A, 4B), potentially contributing to the additional GFAP phosphorylation sites observed in AD aggregates from ApoE(4,4) vs. ApoE(3,3) individuals (FIG. 1B, 1C).

Computational screening identifies novel small molecules predicted to bindstably to GFAP. To identify novel GFAP-specific inhibitors, we screened structures from the ChemBridge library, comprising ˜750,000 small molecules, by in silico docking simulations. For target-based docking we chose the predicted druggable pocket in the transitional GFAP structure (FIG. 2C, at 200 ns). To improve predictive throughput, we conducted computational screening in three stages, increasing the docking stringencies at each stage (see Methods for details), within the Schrodinger Glide docking module (Balasubramaniam et al., 2020). We first performed virtual docking of the entire ChemBridge library in high-throughput mode, followed by redocking of the top 1% of lead molecules in standard-precision mode. The top 1% of molecules emerging from stage 2 (74 structures) were analyzed for implicit-solvent-based free energy of interaction, using Schrodinger's MM-GBSA module (Balasubramaniam et al., 2020). The molecules predicted to have the lowest ΔG_{binding} to GFAP were then pursued for their impact on protein aggregation in vivo. This three-stage procedure for simulated docking produced a set of molecules predicted to bind most avidly and stably to the GFAP target pocket. The three best candidates (labeled MSR1, MSR2, and MSR3) were predicted to have ΔG_{binding} surpassing −46 kcal/mol, and to fit well within the modeled GFAP pocket (FIG. 2C). Counter-screening for binding to tubulin predicted negligible affinity (ΔG_{binding}≥−7 kcal/mol) of these drugs for a or p tubulins, or to oligomers of a and p tubulin, although most of the top-ranked drugs were projected to bind tubulin as avidly as they bound GFAP (data not shown).

Top-ranked drugs suppress in vitro and in vivo aggregation as effectively as knockdown of GFAP, and suppress the same co-aggregate constituents. Three lead compounds with the lowest predicted ΔG_{binding}values (FIG. 5A) were pursued for experimental validation in a human cell-culture model of neurodegenerative amyloidosis, and in C. elegans whole-animal models of AD-like aggregation. MSR3 was found in initial testing to be cytotoxic to neuroblastoma cells and to delay C. elegans development at all doses tested (data not shown), and therefore was not pursued. MSR1 and MSR2 were initially tested for in vivo efficacy in SH-SYSY-APP_{Sw} neuroblastoma cells; aggregation in this model was especially well suppressed by MSR1 (FIGS. 5B and 5C). In all assays, MSR1 was superior or similar in efficacy to MSR2. FIG. 5B shows SH-SYSY-APP_{Sw} neuroblastoma cells stained for amyloid with thioflavin T, after exposure to vehicle (control) or MSR1; in multiple experiments, thioflavin fluorescence declined approximately 2-fold in MSR1-treated cells. Total sarkosyl-insoluble aggregate proteins were isolated and separated on acrylamide-SDS gels, and protein was then stained with SYPRO Ruby. Gel lanes (FIG. 5D, left to right) show vehicle-treated control cells, cells treated 48 h with short interfering RNA (siRNA) targeting GFAP, or cells treated 48 h with MSR1 or MSR2, drugs predicted to bind stably and selectively to GFAP. GFAP siRNA suppressed aggregate protein by 65-80%, while MSR1 provided 60-75% suppression, but MSR2 did not significantly reduce the amount of aggregate protein.

Proteomic identification of proteins in sarkosyl-insoluble aggregates from SH-SY5Y-APP_{Sw} cells revealed that proteins present in sarkosyl-insoluble aggregates of control cells (>7 spectral counts) but completely eliminated by MSR1 treatment (0 hits) coincided remarkably well (87% concordance) with those eliminated by siRNA knock-down of GFAP (FIG. 5E), whereas 4 proteins (CBX8, TSPYL5, CDK2, and KRT33B) were found to be substantially upregulated by either treatment. The excluded group includes a number of proteins involved in microtubule assembly and/or interactions: plectin, dynactin-1, synapsin-1, ankyrin B, MAP1A, MAP2, and α-tubulin. The effects of treatment with MSR1 vs. GFAP siRNA are well correlated (R=0.77, P≤3E-280; FIG. 5F). The observation of such highly concordant and correlated effects of MSR1 and GFAP RNAi on aggregate composition provides compelling evidence that GFAP is the chief functional target through which MSR1 lowers aggregation.

Drugs MSR1 and MSR2 were also tested in several C. elegans models of human neurodegenerative diseases. In a tauopathy model strain (VH255) expressing normal human tau in C. elegans muscle, its aggregation caused paralysis (failure to move in response to prodding) but was alleviated to a similar extent by treatment with 1-μM MSR1 or siRNA against ifp-1, the closest nematode homolog of GFAP (FIG. 7D). MSR1 also effected significant rescue of chemotaxis in a C. elegans model of neuronal amyloidosis (CL2355), in which migration toward a chemo-attractant is impaired by pan-neuronal expression of human Aβ₄₂. Addition of MSR1 at 0.1 μM restored chemotaxis to ˜90% (FIG. 7e), the same level as in wild-type or uninduced worms (not shown). Fluorescent muscle aggregates accumulate with age in a strain expressing Q40::YFP in muscle, mimicking the threshold polyglutamine-array expansion for huntingtin protein, i.e. a length sufficient to elicit symptoms of Huntington's disease in people, and paralysis in nematodes. FIG. 7F shows aggregate intensity per worm at 5 days of age post-hatch, reduced ˜50% by MSR1 at 10 μM, vs. 35% at 0.1-μM (each treatment differing from vehicle-only controls at P≤0.005).

### DISCUSSION

Aging is the most influential non-genetic risk factor for a variety of dementias, and also for many other adult-onset diseases that impose significant burdens on aging adults and on our healthcare system (Niccoli and Partridge, 2012). Neurodegenerative diseases such as AD, Parkinson's, and amyotrophic lateral sclerosis (ALS), as well as conditions as diverse as hypertension (Ayyadevara et al., 2016d), sarcopenia (Ayyadevara et al., 2016c), and even several cancers (Ano Bom et al., 2012), all show accrual of distinctive aggregate foci featuring disease-specific “diagnostic” proteins. We have identified many proteins within immunopurified aggregate subsets (Ayyadevara et al., 2016b), and identified intra-aggregate protein-protein interfaces by cross-linking (Balasubramaniam et al., 2019).

GFAP, one of the numerous proteins enriched in AD aggregates relative to age- matched controls (Ayyadevara et al., 2016b), now joins a small set of neuropathology- associated proteins that display disease-specific hyperphosphorylation. These include tau (AD, PD, ALS), Aβ₁₋₄₂ (AD) TDP43 (ALS), and α-synuclein (PD) (Ayyadevara et al., 2016b; Bai et al., 2021; Ferrer et al., 2021; Mavroudis et al., 2020; Sternburg et al., 2021; Xu et al., 2015; Zhang et al., 2019). We identified three GFAP serines that are highly phosphorylated in aggregates isolated from hippocampi of AD(3,3) individuals, and additional serines and a threonine near the N- and C-termini for which phosphorylation is observed only in AD(4,4) aggregates (FIG. 1C). We examined only APOE3 or APOE4 homozygous tissue, but itis reasonable to expect an intermediate outcome for APOE3 E4 heterozygotes.

The sites we observed for GFAP phosphorylation in AD are consistent with the known targets of several kinases previously implicated in AD pathogenesis. These include AKT2 (one of two mammalian AKT paralogs), Rho-associated Kinase 1 (ROCK1), G-protein- coupled Receptor Kinase 2 (BARK/GRK2), and Protein Kinase A (PKA). Altered insulin signaling has been implicated in AD, and AKT is a key downstream effector of the kinase cascade that conveys insulin/insulin like signaling (Chen et al., 2012; Yang et al., 2018; Zheng and Wang, 2021). GRK2 (a.k.a. BARK) was also shown to play a role in development of cardiovascular disease. PKA, a cAMP-dependent kinase, is involved in multiple signaling pathways, is influential in tau hyperphosphorylation, and has been implicated in progression of several neurodegenerative disorders including AD, PD and HD (Carlyle et al., 2014; Dagda et al., 2011; Greggio et al., 2017; Li et al., 2018; Taylor et al., 2021). PKA was also shown to play roles in diabetes (Li et al., 2018) and anxiety- related behavior (Keil et al., 2016).

Knockdowns of several kinases, which could account for the observed GFAP hyperphosphorylations, decreased protein aggregation and associated physiological declines in C. elegans and in human neuroblastoma cells expressing amyloid precursor protein. We note that several of these same kinases have putative target sites in other AD-associated proteins, such as microtubule-associated protein tau, which are expected to further amplify their impact. Multiple AD-promoting targets of these kinases are suggested by results shown in FIG. 3C, 3E, in which some kinase knockdowns provide more effective rescue from AD-like traits than knockdown of GFAP itself. We note, however, that the neuronal efficacy of siRNA knockdowns was not monitored in these experiments, and is typically less in neurons than in other target cells, so the noted comparisons may be misleading.

Based on our data, GFAP is a novel target to relieve aggregate burden in AD and other aggregation-associated diseases. We therefore screened for small molecules that specifically target GFAP, beginning with the 3-dimensional structure of GFAP predicted via powerful fold-recognition and ab initio procedures (Yang et al., 2015). The initial, lowest-ΔG structure includes a small druggable pocket (FIG. 2A). Molecular-dynamic simulations of GFAP structure over time revealed a transition to a metastable state in which the binding cavity was expanded ˜3-fold over its initial volume. The molecular structure of GFAP at 200 nsec (FIG. 2C) was selected as the target for drug screening. Several descriptors (RMSD, RMSF, and pocket volume) were used to monitor GFAP structural change during the simulation.

Proteomics of brain aggregates indicated strikingly differential phosphorylation of GFAP from AD tissue. Although phosphomimetic substitutions are not perfect mimics of actual protein phosphorylation, computer simulations of the predicted AD(3,3) and AD(4,4) structures, incorporating pseudo-phosphorylated sites as observed, were consistent with the hypothesis that phosphorylation status can alter GFAP structural dynamics. Simulation data do not permit us to infer the extent to which phosphorylation of any individual kinase target is responsible for destabilizing the GFAP structure, or might alternatively favor accessibility of subsequently modified kinase sites.

We tested the top 3 candidates emerging from 3 successive screens for GFAP binding, plus a counter-screen to eliminate tubulin-binding drugs, for efficacy in a variety of aggregation-model systems. Of these, MSR1 displayed efficacy close to that of GFAP knockdown (using RNAi to GFAP or its closest nematode homolog) in each assay, and proteomic analysis of aggregates revealed a near-identical set of proteins depleted or eliminated from aggregates.

These results demonstrate the impact of GFAP and its predicted kinases on protein aggregation in human-cell and C. elegans models of neuropathic aggregation, and in vivo demonstrations of its anti-aggregative efficacy.

### REFERENCES

Ano Bom, A. P., Rangel, L. P., Costa, D. C., de Oliveira, G. A., Sanches, D., Braga, C. A., Gava, L. M., Ramos, C. H., Cepeda, A. O., Stumbo, A. C., et al. (2012). Mutant p53 aggregates into prion-like amyloid oligomers and fibrils: implications for cancer. J Biol Chem 287, 28152-28162.
Ayyadevara, S., Balasubramaniam, M., Gao, Y., Yu, L. R., Alla, R., and Shmookler Reis, R. (2015). Proteins in aggregates functionally impact multiple neurodegenerative disease models by forming proteasome- blocking complexes. Aging Cell 14, 35-48.
Ayyadevara, S., Balasubramaniam, M., Johnson, J., Alla, R., Mackintosh, S. G., and Shmookler Reis, R.J. (2016a). PIP3-binding proteins promote age-dependent protein aggregation and limit survival in C. elegans. Oncotarget 7, 48870-48886.
Ayyadevara, S., Balasubramaniam, M., Kakraba, S., Alla, R., Mehta, J. L., and Shmookler Reis, R. J. (2017). Aspirin-Mediated Acetylation Protects Against Multiple Neurodegenerative Pathologies by Impeding Protein Aggregation. Antioxid Redox Signal 27, 1383-1396.
Ayyadevara, S., Balasubramaniam, M., Parcon, P. A., Barger, S. W., Griffin, W. S., Alla, R., Tackett, A. J., Mackintosh, S. G., Petricoin, E., Zhou, W., et al. (2016b). Proteins that mediate protein aggregation and cytotoxicity distinguish Alzheimer's hippocampus from normal controls. Aging Cell 15, 924-939.
Ayyadevara, S., Balasubramaniam, M., Suri, P., Mackintosh, S. G., Tackett, A. J., Sullivan, D. H., Shmookler Reis, R. J., and Dennis, R.A. (2016c). Proteins that accumulate with age in human skeletal-muscle aggregates contribute to declines in muscle mass and function in Caenorhabditis elegans. Aging (Albany NY) 8, 3486-3497.
Ayyadevara, S., Mercanti, F., Wang, X., Mackintosh, S. G., Tackett, A. J., Prayaga, S. V., Romeo, F., Shmookler Reis, R. J., and Mehta, J. L. (2016d). Age- and Hypertension-Associated Protein Aggregates in Mouse Heart Have Similar Proteomic Profiles. Hypertension 67, 1006-1013.
Bai, B., Vanderwall, D., Li, Y., Wang, X., Poudel, S., Wang, H., Dey, K. K., Chen, P. C., Yang, K., and Peng, J. (2021). Proteomic landscape of Alzheimer's Disease: novel insights into pathogenesis and biomarker discovery. Mol Neurodegener 16, 55.
Balasubramaniam, M., Ayyadevara, S., and Shmookler Reis, R. J. (2018). Structural insights into pro- aggregation effects of C. elegans CRAM-1 and its human ortholog SERF2. Sci Rep 8, 14891.
Balasubramaniam, M., Mainali, N., Bowroju, S. K., Atluri, P., Penthala, N. R., Ayyadevera, S., Crooks, P. A., and Shmookler Reis, R. J. (2020). Structural modeling of GSK3beta implicates the inactive (DFG-out) conformation as the target bound by TDZD analogs. Sci Rep 10, 18326.
Banerjee, T. D., Reihl, K., Swain, M., Torres, M., and Dagda, R. K. (2021). Mitochondrial PKA Is Neuroprotective in a Cell Culture Model of Alzheimer's Disease. Mol Neurobiol 58, 3071-3083.
Bartl, M., Dakna, M., Galasko, D., Hutten, S. J., Foroud, T., Quan, M., Marek, K., Siderowf, A., Franz, J., Trenkwalder, C., et al. (2021). Biomarkers of neurodegeneration and glial activation validated in Alzheimer's disease assessed in longitudinal cerebrospinal fluid samples of Parkinson's disease. PLoS One 16, e0257372.
Brandt, R., Gergou, A., Wacker, I., Fath, T., and Hutter, H. (2009). A Caenorhabditis elegans model of tau hyperphosphorylation: induction of developmental defects by transgenic overexpression of Alzheimer's disease-like modified tau. Neurobiol Aging 30, 22-33.
Brodie, C., Kuperstein, I., Acs, P., and Blumberg, P.M. (1998). Differential role of specific PKC isoforms in the proliferation of glial cells and the expression of the astrocytic markers GFAP and glutamine synthetase. Brain Res Mol Brain Res 56, 108-117.
Calabrese, V., Santoro, A., Monti, D., Crupi, R., Di Paola, R., Latteri, S., Cuzzocrea, S., Zappia, M., Giordano, J., Calabrese, E. J., et al. (2018). Aging and Parkinson's Disease: Inflammaging, neuroinflammation and biological remodeling as key factors in pathogenesis. Free Radic Biol Med 115, 80-91.
Carlyle, B. C., Nairn, A. C., Wang, M., Yang, Y., Jin, L. E., Simen, A. A., Ramos, B. P., Bordner, K. A., Craft, G. E., Davies, P., et al. (2014). cAMP-PKA phosphorylation of tau confers risk for degeneration in aging association cortex. Proc Natl Acad Sci USA 111, 5036-5041.
Chen, L. M., Xiong, Y. S., Kong, F. L., Qu, M., Wang, Q., Chen, X. Q., Wang, J. Z., and Zhu, L. Q. (2012). Neuroglobin attenuates Alzheimer-like tau hyperphosphorylation by activating Akt signaling. J Neurochem 120, 157-164.
Dagda, R. K., Gusdon, A. M., Pien, I., Strack, S., Green, S., Li, C., Van Houten, B., Cherra, S. J., 3rd, and Chu, C. T. (2011). Mitochondrially localized PKA reverses mitochondrial pathology and dysfunction in a cellular model of Parkinson's disease. Cell Death Differ 18, 1914-1923.
Dani, M., Wood, M., Mizoguchi, R., Fan, Z., Walker, Z., Morgan, R., Hinz, R., Biju, M., Kuruvilla, T., Brooks, D. J., et al. (2018). Microglial activation correlates in vivo with both tau and amyloid in Alzheimer's disease. Brain 141, 2740-2754.
de Souza, R. F., Augusto, R. L., de Moraes, S. R. A., de Souza, F. B., Goncalves, L., Pereira, D. D., Moreno, G. M. M., de Souza, F. M. A., and Andrade-da-Costa, B. (2020). Ultra-Endurance Associated With Moderate Exercise in Rats Induces Cerebellar Oxidative Stress and Impairs Reactive GFAP Isoform Profile. Front Mol Neurosci 13, 157.
Donat, C. K., Scott, G., Gentleman, S. M., and Sastre, M. (2017). Microglial Activation in Traumatic Brain Injury. Front Aging Neurosci 9, 208.
Dosanjh, L. E., Brown, M. K., Rao, G., Link, C. D., and Luo, Y. (2010). Behavioral phenotyping of a transgenic Caenorhabditis elegans expressing neuronal amyloid-beta. J Alzheimers Dis 19, 681-690.
Dostal, V., and Link, C. D. (2010). Assaying beta-amyloid toxicity using a transgenic C. elegans model. J Vis Exp.
Drummond, E., Nayak, S., Pires, G., Ueberheide, B., and Wisniewski, T. (2018). Isolation of Amyloid Plaques and Neurofibrillary Tangles from Archived Alzheimer's Disease Tissue Using Laser-Capture Microdissection for Downstream Proteomics. Methods Mol Biol 1723, 319-334.
Fan, Y., and He, J. J. (2016). HIV-1 Tat Induces Unfolded Protein Response and Endoplasmic Reticulum Stress in Astrocytes and Causes Neurotoxicity through Glial Fibrillary Acidic Protein (GFAP) Activation and Aggregation. J Biol Chem 291, 22819-22829.
Ferrer, I., Andres-Benito, P., Ausin, K., Pamplona, R., Del Rio, J. A., Fernandez-Irigoyen, J., and Santamaria, E. (2021). Dysregulated protein phosphorylation: A determining condition in the continuum of brain aging and Alzheimer's disease. Brain Pathol, e12996.
Fire, A., Albertson, D., Harrison, S. W., and Moerman, D. G. (1991). Production of antisense RNA leads to effective and specific inhibition of gene expression in C. elegans muscle. Development 113, 503-514.
Fire, A., Xu, S., Montgomery, M. K., Kostas, S. A., Driver, S. E., and Mello, C.C. (1998). Potent and specific genetic interference by double-stranded RNA in Caenorhabditis elegans. Nature 391, 806-811.
Greggio, E., Bubacco, L., and Russo, I. (2017). Cross-talk between LRRK2 and PKA: implication for Parkinson's disease?Biochem Soc Trans 45, 261-267.
Henderson, B. W., Gentry, E. G., Rush, T., Troncoso, J. C., Thambisetty, M., Montine, T. J., and Herskowitz, J. H. (2016). Rho-associated protein kinase 1 (ROCK1) is increased in Alzheimer's disease and ROCK1 depletion reduces amyloid-beta levels in brain. J Neurochem 138, 525-531.
Hoenig, M. C., Willscheid, N., Bischof, G. N., van Eimeren, T., Drzezga, A., and Alzheimer's Disease Neuroimaging, I. (2020). Assessment of Tau Tangles and Amyloid-beta Plaques Among Super Agers Using PET Imaging. JAMA Netw Open 3, e2028337.
Hol, E. M., and Capetanaki, Y. (2017). Type III Intermediate Filaments Desmin, Glial Fibrillary Acidic Protein (GFAP), Vimentin, and Peripherin. Cold Spring Harb Perspect Biol 9.
Ishigami, A., Masutomi, H., Handa, S., Nakamura, M., Nakaya, S., Uchida, Y., Saito, Y., Murayama, S., Jang, B., Jeon, Y. C., et al. (2015). Mass spectrometric identification of citrullination sites and immunohistochemical detection of citrullinated glial fibrillary acidic protein in Alzheimer's disease brains. J Neurosci Res 93, 1664-1674.
Ishiki, A., Kamada, M., Kawamura, Y., Terao, C., Shimoda, F., Tomita, N., Arai, H., and Furukawa, K. (2016). Glial fibrillar acidic protein in the cerebrospinal fluid of Alzheimer's disease, dementia with Lewy bodies, and frontotemporal lobar degeneration. J Neurochem 136, 258-261.
Jin, Z., Fu, Z., Yang, J., Troncosco, J., Everett, A. D., and Van Eyk, J. E. (2013). Identification and characterization of citrulline-modified brain proteins by combining HCD and CID fragmentation. Proteomics 13, 2682-2691.
Kakraba, S., Ayyadevara, S., Penthala, N. R., Balasubramaniam, M., Ganne, A., Liu, L., Alla, R., Bommagani, S. B., Barger, S.W., Griffin, W. S. T., et al. (2019). A Novel Microtubule-Binding Drug Attenuates and Reverses Protein Aggregation in Animal Models of Alzheimer's Disease. Frontiers in Molecular Neuroscience 12.
Kamphuis, W., Mamber, C., Moeton, M., Kooijman, L., Sluijs, J. A., Jansen, A. H., Verveer, M., de Groot, L. R., Smith, V. D., Rangarajan, S., et al. (2012). GFAP isoforms in adult mouse brain with a focus on neurogenic astrocytes and reactive astrogliosis in mouse models of Alzheimer disease. PLoS One 7, e42823.
Keil, M. F., Briassoulis, G., Stratakis, C. A., and Wu, T. J. (2016). Protein Kinase A and Anxiety-Related Behaviors: A Mini-Review. Front Endocrinol (Lausanne) 7, 83.
Ko, H. J., Chiou, S. J., Wong, Y. H., Wang, Y. H., Lai, Y., Chou, C. H., Wang, C., Loh, J. K., Lieu, A. S., Cheng, J. T., et al. (2019). GSKIP-Mediated Anchoring Increases Phosphorylation of Tau by PKA but Not by GSK3beta via cAMP/PKA/GSKIP/GSK3/Tau Axis Signaling in Cerebrospinal Fluid and iPS Cells in Alzheimer Disease. J Clin Med 8.
Laping, N. J., Teter, B., Nichols, N. R., Rozovsky, I., and Finch, C. E. (1994). Glial fibrillary acidic protein: regulation by hormones, cytokines, and growth factors. Brain Pathol 4, 259-275.
Lazarus, R. C., Buonora, J. E., Flora, M. N., Freedy, J. G., Holstein, G. R., Martinelli, G. P., Jacobowitz, D. M., and Mueller, G. P. (2015). Protein Citrullination: A Proposed Mechanism for Pathology in Traumatic Brain Injury. Front Neurol 6, 204.
Lee, S. H., Nam, T. S., Kim, K. H., Kim, J. H., Yoon, W., Heo, S. H., Kim, M. J., Shin, B. A., Perng, M. D., Choy, H. E., et al. (2017). Aggregation-prone GFAP mutation in Alexander disease validated using a zebrafish model. BMC Neurol 17, 175.
Li, H., Yang, S., Wu, J., Ji, L., Zhu, L., Cao, L., Huang, J., Jiang, Q., Wei, J., Liu, M., et al. (2018). cAMP/PKA signaling pathway contributes to neuronal apoptosis via regulating IDE expression in a mixed model of type 2 diabetes and Alzheimer's disease. J Cell Biochem 119, 1616-1626.
Liu, D., Liu, C., Li, J., Azadzoi, K., Yang, Y., Fei, Z., Dou, K., Kowall, N. W., Choi, H. P., Vieira, F., et al. (2013). Proteomic analysis reveals differentially regulated protein acetylation in human amyotrophic lateral sclerosis spinal cord. PLoS One 8, e80779.
Liu, L., Li, Y., Van Eldik, L. J., Griffin, W. S., and Barger, S. W. (2005). S100B-induced microglial and neuronal IL-1 expression is mediated by cell type-specific transcription factors. J Neurochem 92, 546-553.
Mavroudis, I., Petridis, F., Chatzikonstantinou, S., and Kazis, D. (2020). Alpha-synuclein Levels in the Differential Diagnosis of Lewy Bodies Dementia and Other Neurodegenerative Disorders: A Meta-analysis. Alzheimer Dis Assoc Disord 34, 220-224.
Moeton, M., Stassen, O. M., Sluijs, J. A., van der Meer, V. W., Kluivers, L. J., van Hoorn, H., Schmidt, T., Reits, E. A., van Strien, M. E., and Hol, E. M. (2016). GFAP isoforms control intermediate filament network dynamics, cell morphology, and focal adhesions. Cell Mol Life Sci 73, 4101-4120.
Morley, J. F., Brignull, H. R., Weyers, J. J., and Morimoto, R. I. (2002). The threshold for polyglutamine-expansion protein aggregation and cellular toxicity is dynamic and influenced by aging in Caenorhabditis elegans. Proc Natl Acad Sci USA 99, 10417-10422.
Muccigrosso, M. M., Ford, J., Benner, B., Moussa, D., Burnsides, C., Fenn, A. M., Popovich, P. G., Lifshitz, J., Walker, F. R., Eiferman, D. S., et al. (2016). Cognitive deficits develop lmonth after diffuse brain injury and are exaggerated by microglia-associated reactivity to peripheral immune challenge. Brain Behav Immun 54, 95-109.
Nawashiro, H., Messing, A., Azzam, N., and Brenner, M. (1998). Mice lacking GFAP are hypersensitive to traumatic cerebrospinal injury. Neuroreport 9, 1691-1696.
Niccoli, T., and Partridge, L. (2012). Ageing as a risk factor for disease. Curr Biol 22, R741-752.
Obrenovich, M. E., Morales, L. A., Cobb, C. J., Shenk, J. C., Mendez, G. M., Fischbach, K., Smith, M. A., Qasimov, E. K., Perry, G., and Aliev, G. (2009a). Insights into cerebrovascular complications and Alzheimer disease through the selective loss of GRK2 regulation. J Cell Mol Med 13, 853-865.
Obrenovich, M. E., Palacios, H. H., Gasimov, E., Leszek, J., and Aliev, G. (2009b). The GRK2 Overexpression Is a Primary Hallmark of Mitochondrial Lesions during Early Alzheimer Disease. Cardiovasc Psychiatry Neurol 2009, 327360.
Obrenovich, M. E., Smith, M. A., Siedlak, S. L., Chen, S. G., de la Torre, J. C., Perry, G., and Aliev, G. (2006). Overexpression of GRK2 in Alzheimer disease and in a chronic hypoperfusion rat model is an early marker of brain mitochondrial lesions. Neurotox Res 10, 43-56.
Russo, I. (2019). The role of LRRK2 on PKA-NFkappaB pathway in microglia cells: implications for Parkinson's disease. Neural Regen Res 14, 1713-1714.
Schulz, I., Kruse, N., Gera, R. G., Kremer, T., Cedarbaum, J., Barbour, R., Zago, W., Schade, S., Otte, B., Bartl, M., et al. (2021). Systematic Assessment of 10 Biomarker Candidates Focusing on alpha-Synuclein-Related Disorders. Mov Disord.
Sternburg, E. L., Gruijs da Silva, L. A., and Dormann, D. (2021). Post-translational modifications on RNA-binding proteins: accelerators, brakes, or passengers in neurodegeneration?Trends Biochem Sci.
Streit, W. J., Mrak, R. E., and Griffin, W. S. (2004). Microglia and neuroinflammation: a pathological perspective. J Neuroinflammation 1, 14.
Taylor, S. S., Wallbott, M., Machal, E. M. F., Soberg, K., Ahmed, F., Bruystens, J., Vu, L., Baker, B., Wu, J., Raimondi, F., et al. (2021). PKA Cbeta: a forgotten catalytic subunit of cAMP-dependent protein kinase opens new windows for PKA signaling and disease pathologies. Biochem J 478, 2101-2119.
Thomsen, R., Daugaard, T. F., Holm, I. E., and Nielsen, A. L. (2013). Alternative mRNA splicing from the glial fibrillary acidic protein (GFAP) gene generates isoforms with distinct subcellular mRNA localization patterns in astrocytes. PLoS One 8, e72110.
Xu, Y., Deng, Y., and Qing, H. (2015). The phosphorylation of alpha-synuclein: development and implication for the mechanism and therapy of the Parkinson's disease. J Neurochem 135, 4-18.
Yang, J., Yan, R., Roy, A., Xu, D., Poisson, J., and Zhang, Y. (2015). The I-TASSER Suite: protein structure and function prediction. Nat Methods 12, 7-8.
Yang, L., Wang, H., Liu, L., and Xie, A. (2018). The Role of Insulin/IGF-1/PI3K/Akt/GSK3beta Signaling in Parkinson's Disease Dementia. Front Neurosci 12, 73.
Zhang, L., Chen, Y., Liu, M., Wang, Y., and Peng, G. (2019). TDP-43 and Limbic-Predominant Age-Related TDP- 43 Encephalopathy. Front Aging Neurosci 11, 376.
Zhang, Z., Shen, Q., Wu, X., Zhang, D., and Xing, D. (2020). Activation of PKA/SIRT1 signaling pathway by photobiomodulation therapy reduces Abeta levels in Alzheimer's disease models. Aging Cell 19, e13054.
Zheng, M., and Wang, P. (2021). Role of insulin receptor substance-1 modulating PI3K/Akt insulin signaling pathway in Alzheimer's disease. 3 Biotech 11, 179.

1. A method reducing aggregate protein abundance in a protein aggregate, the method comprising administering an effective amount of a compound that stably binds a Glial Fibrillary Acidic Protein (GFAP) to a subject, wherein the aggregate protein comprises BSN, SYN1, MAP2, PLEC, RAB10, MAP1A, DCTN, TUBA4A, SPART, PRKDC, or any combination thereof.

2. The method of claim 1, wherein the subject suffers from a neurodegenerative disorder.

3. The method of claim 2, wherein the neurodegenerative disorder is Alzheimer's disease (AD).

4. The method of claim 1, wherein the compound is MSR1.

5. The method of claim 1, wherein GFAP is a phosphorylated GFAP.

6. The method of claim 1, wherein the compound has negligible affinity for α-tubulin, β-tubulin, or an oligomer thereof.

7. The method of claim 1, wherein the compound has a ΔG_{binding} to GFAP of less than −40 kcal/mol.

8. The method of claim 1, wherein of the aggregate protein comprises BSN, SYN1, MAP2, PLEC, RAB10, MAP1A, DCTN, TUBA4A, SPART, and PRKDC.

9. A method for the treatment of a subject suffering from a neurodegenerative disorder, the method comprising administering an effective amount of a compound that stably binds a GFAP.

10. The method of claim 9, wherein the compound is MSR1.

11. The method of claim 9, wherein the neurodegenerative disease is AD.

12. The method of claim 9, wherein the GFAP is a phosphorylated GFAP.

13. The method of claim 9, wherein the compound has negligible affinity for α-tubulin, β-tubulin, or an oligomer thereof.

14. The method of claim 9, wherein the compound has a ΔG_{binding} less than −50 kcal/mol.

15. A method for increasing aggregate protein abundance in a protein aggregate, the method comprising administering an effective amount of a compound that stably binds a Glial Fibrillary Acidic Protein (GFAP) to a subject, wherein the aggregate protein comprises CBX8, TSPYL5, CDK2, KRT33B, or any combination thereof.

16. The method of claim 15, wherein the subject suffers from a neurodegenerative disorder.

17. The method of claim 16, wherein the neurodegenerative disorder is Alzheimer's disease (AD).

18. The method of claim 15, wherein the compound is MSR1.

19. The method of claim 15, wherein GFAP is a phosphorylated GFAP.

20. The method of claim 15, wherein the compound has negligible affinity for α-tubulin, β-tubulin, or an oligomer thereof.

21. The method of claim 15, wherein the compound has a ΔG_{binding}less than −40 kcal/mol.

22. The method of claim 15, wherein the aggregate protein comprises CBX8, TSPYL5, CDK2, and KRT33B.
